(19) United States
(12) Patent Application Publication
(10) Pub. No.: US 2024/0415397 A1
(43) Pub. Date: Dec. 19, 2024

(54) INDWELLING VASCULAR PROBE FOR BLOOD PARAMETER SENSING

(71) Applicant: Becton, Dickinson and Company, Franklin Lakes, NJ (US)

(72) Inventors: Jonathan Karl Burkholz, Salt Lake City, UT (US); Joseph Spataro, Cottonwood Heights, UT (US); Kyle Thornley, Farmington, UT (US)

(21) Appl. No.: 18/677,627

(22) Filed: May 29, 2024

Related U.S. Application Data
(60) Provisional application No. 63/521,497, filed on Jun. 16, 2023.

(51) Int. Cl. A61B 5/0205; A61B 5/00

(52) U.S. Cl. CPC A61B 5/02055; A61B 5/6848; A61B 5/6876; A61B 5/742; A61B 2560/0462; A61B 2560/063

(57) ABSTRACT

A vascular probe insertion assembly may include a slotted cannula having a sharpened tip configured to be inserted into a patient's vascular anatomy and a sidewall extending proximally from the sharpened tip, the sidewall defining a slot. The vascular probe insertion assembly may include a splitable sheath shaped to encircle at least part of the sidewall and a digital probe comprising a sensor tip configured to generate sensor data indicative of operation of the vascular anatomy and a shaft extending proximally of the sensor tip. The vascular probe insertion assembly may include a data hub operatively coupled to the digital probe. The data hub may be configured to receive the sensor data and convey the sensor data to a monitoring system.

### RELATED APPLICATIONS

This application claims the benefit of U.S. Provisional Patent Application No. 63/521,497, which was filed on Jun. 16, 2023, which is incorporated herein in its entirety.

### BACKGROUND

Arterial catheters and systems provide health care professionals with a method of monitoring a patient's arterial hemodynamic parameters as well as provide access for collecting arterial blood for arterial blood gas (ABG) testing and analysis. Currently arterial catheters devices, systems, and methods, however, may have significant blood exposure risks and other performance issues such as accurate placement of the arterial catheter. Still further, current hemodynamic monitoring and ABG collection systems can be very complicated, expensive, and may require significant amounts of time and resources in order to collect the necessary samples. Still further a significant amount of time and resources may be necessary in order to maintain the arterial line so that risks of complications such as infections and catheter-related blood stream infection (BRBSI) are minimized. Even further, a significant amount of time and resources may be necessary in order to ensure proper line and device flushing as well as preserving arterial blood, among other processes associated with currently-implemented arterial catheters.

The subject matter claimed in the present disclosure is not limited to embodiments that solve any disadvantages or that operate only in environments such as those described in the present disclosure. Rather, this background is provided to describe an environment in which the presently described embodiments may operate.

### SUMMARY

The present disclosure relates generally to vascular probe insertion assembly used to insert a digital probe into the vascular anatomy of a patient. In embodiments described in the present disclosure, the vascular probe may include a slotted cannula for insertion of the vascular probe into a patient's vascular anatomy. This slotted cannula may have a slot formed along the length of the slotted cannula so that a digital probe may be placed coaxially therein. In some embodiments, a splitable sheath may be formed coaxially outside of the slotted cannula to keep the digital probe within the slotted cannula. After insertion of the cannula into a patient's vascular anatomy, a data hub operatively coupled to the digital probe may be affixed to the patient's body so that the digital probe is maintained within the patient's vascular anatomy.

In some embodiments, a needle hub may be operatively coupled to the slotted cannula. The needle hub may include a fluid reservoir to determine when the cannula has been inserted into the patient's vascular anatomy.

In some embodiments, the vascular probe includes a needle safety shield including a via through which the cannula may pass. The needle safety shield may be operatively coupled with the data hub. In some embodiments, when the cannula is withdrawn proximally the cannula may slide with respect to the needle safety shield, and the needle safety shield may cover the bevel when the cannula is removed from the patient's vascular anatomy. In some embodiments, the needle safety shield includes a needle safety shield cutting blade formed within the via formed through the needle safety shield to cut the splitable sheath when the vascular probe is retracted from within the patient's vascular anatomy.

In some embodiments, the data hub includes a wireless transmitter formed within the data hub to wirelessly transmit data received at the digital probe within the patient's vascular anatomy to a vascular monitoring system. In some embodiments, the vascular probe includes contact pins formed at a proximal end of the data hub to interface with a wired connection used to operatively couple the data hub to a vascular monitoring system.

In some embodiments, the vascular probe may include devices used to securely insert the cannula into the patient's anatomy as well as hold the data hub to the patient's body (e.g., an arm). In some embodiments, the vascular probe may include a syringe operatively coupled to a proximal end of the slotted cannula to provide stabilized insertion of the slotted cannula into the patient's vascular anatomy. In some embodiments, the vascular probe may include a stabilization platform operatively coupled to the data hub to secure the data hub to an exterior surface of the patient's anatomy as the digital probe is maintained within the patient's vascular anatomy.

The present specification also describes a method of manufacturing a vascular probe. In some embodiments, the method may include inserting a digital probe into a slotted cannula. In some embodiments, the digital probe may be used to detect parameters of a patient's vascular anatomy. In some embodiments, the method may include forming a needle safety shield around the slotted cannula by passing the slotted cannula through a via formed through the needle safety shield. In some embodiments, the method may include forming a splitable sheath around the slotted cannula to secure the digital probe into the slotted cannula during insertion into the vascular anatomy of a patient and operatively coupling a data hub to the digital probe.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is a side perspective view of a vascular probe insertion assembly according to some embodiments of the present disclosure;

FIG. 2 is a side perspective view of a vascular probe insertion assembly according to some embodiments of the present disclosure;

FIG. 3 is a side perspective view of a needle safety shield of a vascular probe insertion assembly according to some embodiments of the present disclosure;

FIG. 4 is a perspective view of a digital probe of a vascular probe insertion assembly accessing a vascular anatomy according to some embodiments of the present disclosure;

FIG. 5 is a perspective view of a vascular probe insertion assembly according to some embodiments of the present disclosure;

FIG. 6 is a side perspective view of a vascular probe insertion assembly according to some embodiments of the present disclosure;

FIG. 7 is a side elevation view of a data hub and digital probe according to some embodiments of the present disclosure;

FIG. 8 is a top view of a data hub and digital probe according to some embodiments of the present disclosure;

FIG. 9 is a top view of a data hub and digital probe according to some embodiments of the present disclosure;

FIG. 10 is a top view of a digital probe and data hub according to some embodiments of the present disclosure;

FIG. 11 is a side perspective view of a vascular probe insertion assembly according to some embodiments of the present disclosure;

FIG. 12 is a side perspective view of a vascular probe insertion assembly according to some embodiments of the present disclosure;

FIG. 13 is a side perspective view of a digital probe inserted into vascular anatomy according to some embodiments of the present disclosure;

FIG. 14 is a top view of a digital probe and data hub according to some embodiments of the present disclosure;

FIG. 15 is a top view of a digital probe, data hub, and a wired connection according to some embodiments of the present disclosure;

FIG. 16 is a top view of a digital probe, data hub, and a wired connection according to some embodiments of the present disclosure;

FIG. 17 is a perspective view of a vascular probe insertion assembly with a wired connection according to some embodiments of the present disclosure;

FIG. 18 is a side view of a digital probe with a wired connection according to some embodiments of the present disclosure;

FIG. 19 is a top view of a digital probe and data hub according to some embodiments of the present disclosure;

FIG. 20 is a top view of a digital probe and data hub according to some embodiments of the present disclosure;

FIG. 21 is a side perspective view of a vascular probe insertion assembly according to some embodiments of the present disclosure;

FIG. 22 is a side perspective view of a vascular probe insertion assembly according to some embodiments of the present disclosure;

FIG. 23 is a side perspective view of a vascular probe inserted into vascular anatomy according to some embodiments of the present disclosure;

FIG. 24 is a graphic diagram of a vascular probe insertion assembly interfacing with a data processing and cloud-based system according to some embodiments of the present disclosure;

FIG. 25 is a side, cross-sectional, perspective view of a vascular probe insertion assembly according to some embodiments of the present disclosure;

FIG. 26 is a block diagram of a method of manufacturing a vascular probe insertion assembly according to some embodiments of the present disclosure; and

FIG. 27 is a block diagram of a method of inserting a digital probe of a vascular probe insertion assembly into a patient's anatomy according to some embodiments of the present disclosure.

### DESCRIPTION OF EMBODIMENTS

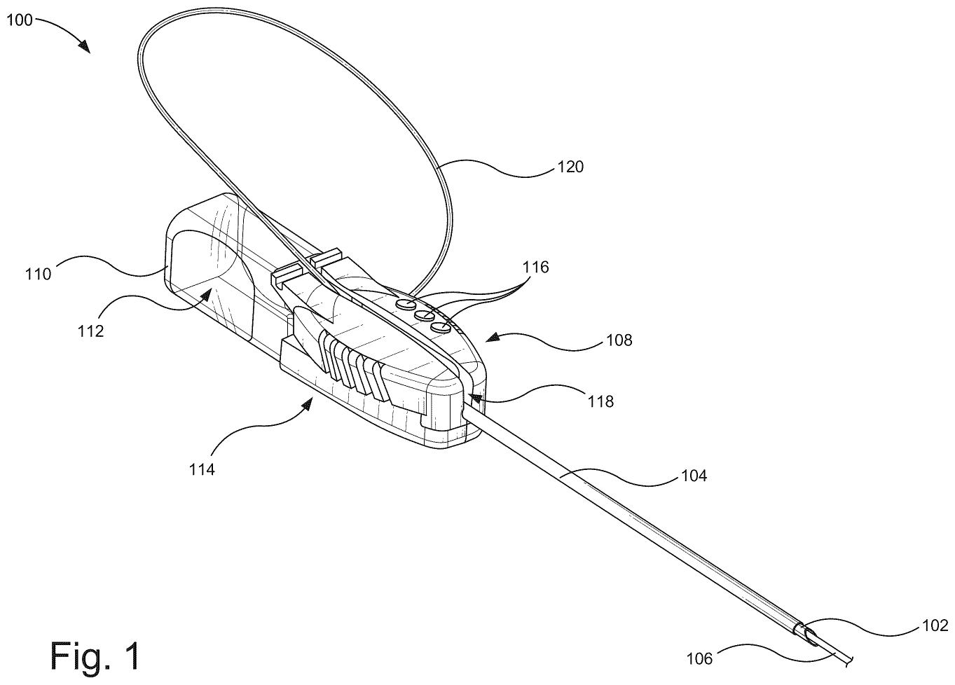

FIG. 1 is a side perspective view of a vascular probe insertion assembly 100 according to some embodiments of the present disclosure. The vascular probe insertion assembly 100 may be used by a doctor, nurse, or other healthcare professional to monitor, detect, and/or measure various bloodstream-based parameters within a patient's vascular anatomy. In the present specification and in the appended claims, the term “vascular anatomy” is understood as any blood vessel or artery where a patient's blood is present. The monitored, detected, and/or measured bloodstream-based parameters may include, for example, venous or arterial blood pressure, temperature, potential of hydrogen (pH) of the patient's blood, presence of organic compounds such as lactate, the presence and amount of oxygen (O₂) in the blood, the percentage of oxygen (SPO₂), as well as other intervascular parameters that may be detected.

The vascular probe insertion assembly 100 may include a number of devices used by a healthcare professional to introduce a digital probe 106 into a vein or artery of a patient. The vascular probe insertion assembly 100 may include a slotted cannula 102. The slotted cannula 102 may include slot formed along the length of the slotted cannula 102 into which a digital probe 106 may be placed. The slot formed in the slotted cannula 102 may form sidewalls extending along the length of the slotted cannula 102. In some embodiments, the sidewalls may maintain the digital probe 106 coaxially or approximately coaxially within the slotted cannula 102 except at the slot.

In some embodiments, the digital probe 106 may, therefore, run coaxially within the slotted cannula 102 until the slotted cannula 102 is removed from the patient's anatomy as described in the present disclosure. In some embodiments, the digital probe 106 may not be coaxial with the slotted cannula 102. In some embodiments, the orientation of the slot formed in the slotted cannula 102 is aligned with a trench or digital probe channel formed through the data hub 108 so that the digital probe 106 can leave the digital probe channel formed in the data hub 108 when the digital probe 106 is inserted into the patient's vascular anatomy. In some embodiments, the slotted cannula 102 may include a sharp point and/or may be constructed of a rigid material to facilitate entry of the slotted cannula 102 and a splittable sheath 104 into vasculature of the patient. In some embodiments, the splitable sheath 104 creates a closed fluid channel around the slotted cannula 102, allowing fluid, such as blood, to flow through the splitable sheath 104 and provide visual and tactile feedback to a user of entry of the vascular probe insertion assembly 100 into the vasculature of a patient.

The digital probe 106 includes, in some embodiments, a core wire for structure and durability during advancement of the digital probe 106 through the patient's vascular anatomy. The core wire may be a nitinol core wire that runs the length of the digital probe 106 either at the center axis of the digital probe 106 or offset from the center axis. In some embodiments, the digital probe 106 includes optical fibers and/or wires connected to one or more sensors placed along the length of the digital probe 106. In some embodiments, a distal end of the digital probe 106 includes an atraumatic tip that reduces the risk of the digital probe 106 inducing damage or complications in the patient's vein or artery.

The digital probe 106 may be coated with a variety of coatings used to improve the performance of the digital probe 106 as well as reduce the risk of complications such as thrombus or probe-related blood stream infections (e.g., sepsis). These coatings may include silicon lubes that may or may not include an antimicrobial additive such as alkanes or saturated hydrocarbons (e.g., CH (x)). The digital probe 106 may be coated with an anti-thrombogenic or anti-microbial coating and/or polymer additives.

In some embodiments, the sensors present on the digital probe 106 may include any number or type of sensors that detect or measure parameters of a patient's vascular anatomy. The sensors may be placed along the digital probe 106 as individual sensors or bundles of sensors. The sensors may include sensors that incorporate technologies that can detect or measure a patient's blood pressure (venous or arterial), blood gases, blood pH levels, and presence of electrolytes among other types of sensors. In some embodiments, the sensors present on the digital probe 106 may be selected to detect or measure other targeted physiological or procedural parameters.

The vascular probe insertion assembly 100 may include the splitable sheath 104 formed around the slotted cannula 102. The splitable sheath 104 may extend the entire length of the slotted cannula 102 except along a beveled portion of the slotted cannula 102. This allows the sharp terminal end of the slotted cannula 102 to penetrate a patient's skin when the healthcare professional accesses the patient's vein or artery. The splitable sheath 104 may also prevent the digital probe 106 from exiting the slot formed along the slotted cannula 102 during insertion of the vascular probe insertion assembly 100 into the patient's vascular anatomy.

The vascular probe insertion assembly 100 may include a needle hub 110 operatively coupled to a proximal end of the slotted cannula 102 opposite the beveled portion of the slotted cannula 102 that forms a sharpened tip configured to be inserted into a patient's vascular anatomy. The needle hub 110 may include a hollow chamber or fluid reservoir 112 fluidically coupled to the interior of the slotted cannula 102. The fluid reservoir 112 may receive an amount of blood as the healthcare professional inserts the slotted cannula 102 into the patient's vascular anatomy. In this way, the healthcare professional may determine if and when the slotted cannula 102 has penetrated a vein or artery of the patient by viewing whether a flow of blood has entered the fluid reservoir 112. The volume of the fluid reservoir 112 may be sufficient to allow the healthcare professional to view this process while also not drawing a significant amount of blood from the patient. Because the fluid reservoir 112 is used to merely detect when the slotted cannula 102 has reached a vein or artery, the fluid reservoir 112 along with the slotted cannula 102 and needle safety shield 114 described in the present disclosure may be disposed of when the digital probe 106 has been introduced into the patient's vascular anatomy and the slotted cannula 102 has been pulled out of the vein or artery.

The vascular probe insertion assembly 100 may include a needle safety shield 114. The needle safety shield 114 is formed around a portion of the slotted cannula 102 and is allowed to move along the shaft of the slotted cannula 102 during operation. In some embodiments, the needle safety shield 114 has a via formed through it such that the slotted cannula 102 may be passed therethrough. Additionally, the needle safety shield 114, in some embodiments, includes a needle safety shield cutting blade (not shown in FIG. 1) that is used to cut the splitable sheath 104 during extraction of the slotted cannula 102 from the patient's vascular anatomy.

The vascular probe insertion assembly 100 may include a data hub 108 operatively coupled to the digital probe 106. The data hub 108 may include any type of circuitry used to receive and transmit data detected by the digital probe 106 when the digital probe 106 has been successfully inserted into the patient's vein or artery. In some embodiments, this circuitry may include a wireless radio or other transmitting device that allows for the wireless transmission of the data detected at the digital probe 106 to be transmitted to, for example, a vascular monitoring system that is described in the present disclosure. Additionally, the data hub 108 may include a power source, a power management unit (PMU), and a microcontroller or other hardware processing device, among other circuitry. In some embodiments, the data hub 108 may include one or more visual indicators 116 used to signal to the healthcare professionals that, for example, the data hub 108 is detecting parameters of a patient's vascular anatomy due to the digital probe 106 being properly inserted into the patient's vascular anatomy. These visual indicators 116 may include a variety of differently colored light emitting diodes (LEDs) that may indicate proper used by the microcontroller in the data hub 108 to determine whether the digital probe 106 has been properly, improperly, or sub-optimally inserted into the patient's anatomy.

During operation of the vascular probe insertion assembly 100, the healthcare professional may address the patient's anatomy and determine a location where the digital probe 106 may be placed within the patient's vein or artery. Once an appropriate injection site has been determined, the healthcare professional may insert the vascular probe insertion assembly 100 and, specifically, the slotted cannula 102 with its digital probe 106 and the splitable sheath 104 into the patient's anatomy in into a vein or artery. At this point, because the slotted cannula 102 is fluidically coupled with the fluid reservoir 112 of the needle hub 110, the healthcare professional may visually detect that the slotted cannula 102 has reached a vein or artery by the presence of blood within the fluid reservoir 112. Because the needle hub 110 is made of a clear plastic, for example, the healthcare professional may visually detect the filling of the fluid reservoir 112.

Once the healthcare professional has detected the presence of blood in the fluid reservoir 112 of the needle hub 110 indicating that the slotted cannula 102 has reached a vein or artery, the healthcare professional may begin to remove the vascular probe insertion assembly 100 from the patient. In some embodiments, the healthcare professional may do this by grasping the needle hub 110 in one hand and the data hub 108/needle safety shield 114 with the other. While holding the data hub 108/needle safety shield 114 in place, the healthcare professional may begin to pull the needle hub 110 away from the patient and the remaining portions of the vascular probe insertion assembly 100. By doing this, the needle safety shield cutting blade (not shown in FIG. 1) of the needle safety shield 114 may begin to cut the splitable sheath 104 as the slotted cannula 102 is pulled through the via formed through the needle safety shield 114. This will continue until the beveled portion of the slotted cannula 102 is safely housed within the via formed through the needle safety shield 114. This process also may include the removal of the data hub 108 from the needle safety shield 114. In some embodiments, the data hub 108 and needle safety shield 114 may be operatively coupled together via a rail system such that they may be slidably removed from each other as the healthcare professional pulls the slotted cannula 102 and splitable sheath 104 through the via formed through the needle safety shield 114.

As the beveled end of the slotted cannula 102 enters the via formed through the needle safety shield 114, the slotted cannula 102 may be prevented from totally being removed from the via in order to prevent the sharp point of the slotted cannula 102 from touching the healthcare professional and potentially harming the healthcare professional. Additionally, the splitable sheath 104, having been cut lengthwise along the length of the slotted cannula 102 may be separated from the slotted cannula 102 and thrown away with the needle hub 110, slotted cannula 102, and needle safety shield 114.

Additionally, as the slotted cannula 102 is pulled out of the patient's vascular anatomy, the digital probe 106 may remain within the patient's vascular anatomy. In some embodiments, a supplemental length of digital probe 120 may be advanced further into the patient's vascular anatomy by the healthcare professional as the slotted cannula 102 is inserted into the vascular anatomy. This allows the advancement of the digital probe 106 further into the patient's vascular anatomy prior to the slotted cannula 102 being removed to, for example, cause the digital probe 106 to remain within the vascular anatomy and preventing the slotted cannula 102 from dragging the digital probe 106 back out of the vascular anatomy. Additionally, while the slotted cannula 102 is being pulled out of the patient's vascular anatomy, the digital probe 106/supplemental length of digital probe 120 may exit the slotted cannula 102 due to the splitable sheath 104 being cut and the slot formed in the slotted cannula 102 serving as an exit for the digital probe 106 to be removed from the slotted cannula 102.

When the slotted cannula 102, splitable sheath 104, needle safety shield 114, and needle hub 110 have been removed from the digital probe 106 and data hub 108, the healthcare professional may determine whether the digital probe 106 has been successfully inserted into the patient's vascular anatomy. This may be done by determining visually whether the digital probe 106 appears to be passing into the patient's anatomy and whether the visual indicators 116 on the data hub 108 indicate proper insertion of the digital probe 106. Where the healthcare professional has determined improper insertion of the digital probe 106, the digital probe 106 may be removed by pulling the digital probe 106 from the patient's vascular anatomy and a new insertion site may be determined using a new vascular probe insertion assembly 100. Where the healthcare professional has determined that the digital probe 106 is properly inserted, the healthcare professional may secure the data hub 108 to the exterior portion of the patient's anatomy (e.g., arm) and apply a securement dressing (not shown in FIG. 1) over the data hub 108/supplemental length of digital probe 120 to prevent dislodgement of the digital probe 106. In some embodiments, the healthcare professional may also apply a skin adhesive at the injection site where the slotted cannula 102 entered the patient's body to secure the digital probe 106 to the patient during indwelling of the digital probe 106.

Where the digital probe 106 and data hub 108 are no longer needed by the healthcare professional to detect or measure parameters of a patient's vascular anatomy, the healthcare professional may remove the digital probe 106 and data hub 108 and throw them away. In some embodiments, the healthcare professional may do this by gripping the data hub 108 and pulling on the coupled digital probe 106 away from the patient's body. This allows the digital probe 106 to be pulled out of the vein or artery, through the remaining portions of the patient's anatomy, and out of the hole formed by the slotted cannula 102 during insertion.

The vascular probe insertion assembly 100 described in the present disclosure may provide for direct in-vascular measure of hemodynamic and blood based critical patient parameters with a minimum footprint on the patient's body. The vascular probe insertion assembly 100 may also allow for continuous or intermittent monitoring without the procedural and workflow difficulties associated with other monitoring systems. Further, the use of the vascular probe insertion assembly 100 described in the present disclosure may not require use of a separate catheter or vascular access device. Still further, the vascular probe insertion assembly 100 may reduce risks associated with other systems used to complete the measurements that the vascular probe insertion assembly 100 can accomplish. The vascular probe insertion assembly 100 described in the present disclosure may be used for short term or long-term monitoring of critical vascular-based parameters while the digital probe 106 is in place within the patient's vein or artery.

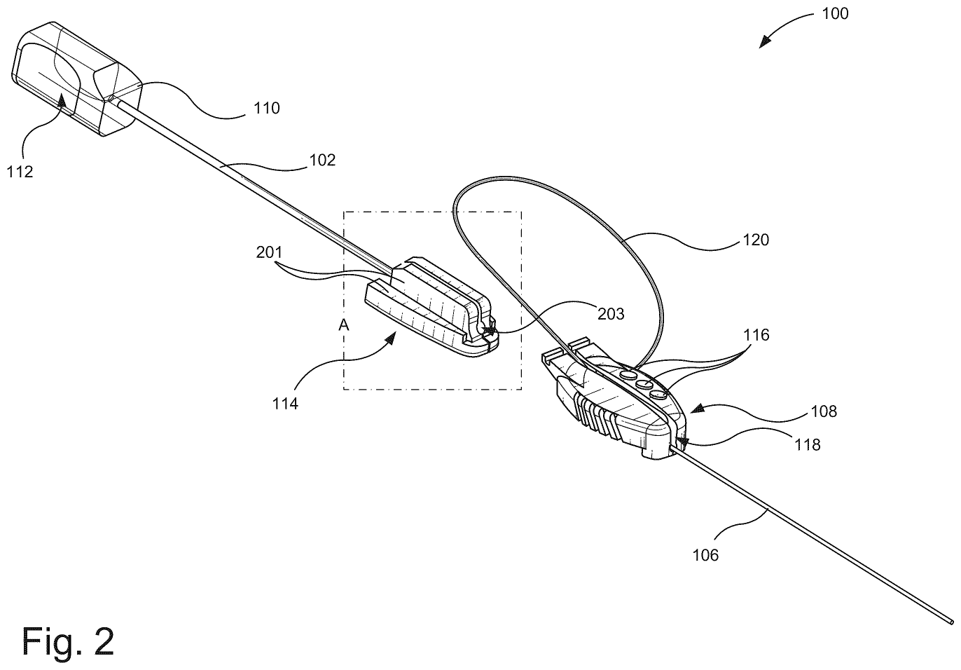

FIG. 2 is a side perspective view of a vascular probe insertion assembly 100 according to some embodiments of the present disclosure. The vascular probe insertion assembly 100 shown in FIG. 2 may be similar to the vascular probe insertion assembly 100 shown in FIG. 1. In the embodiment shown in FIG. 2, the digital probe 106 and data hub 108 have been removed away from the slotted cannula 102, the needle safety shield 114, and the needle hub 110. The splitable sheath 104 shown in FIG. 1 is not shown in FIG. 2 because, as described in the present disclosure, the movement of the needle safety shield 114 towards a distal end of the slotted cannula 102 where the bevel is formed causes a needle safety shield cutting blade of the needle safety shield 114 to cut the splitable sheath 104 away exposing the slotted cannula 102.

As depicted in FIG. 2, the needle safety shield 114 may include a number of attachment surfaces 201 that interface with complementary attachment surfaces formed on the digital probe 106. The attachment surfaces 201 allow the needle safety shield 114 to be operatively coupled, at least temporarily, to the bottom side of the data hub 108 during insertion of the slotted cannula 102 into the vascular anatomy of the patient. As described in the present disclosure, after the slotted cannula 102 has been inserted into the patient's vein or artery, it may be subsequently removed with the digital probe 106 and data hub 108 remaining in place. In the embodiments in the present disclosure, the removal of the slotted cannula 102 causes the digital probe 106 to remain within the patient's vein or artery while the data hub 108 (operatively coupled to the digital probe 106) remains with the patient. By laterally sliding the needle safety shield 114 away from the data hub 108, the needle safety shield 114, the slotted cannula 102, and the needle hub 110 may be removed away from the data hub 108 thereby separating the needle safety shield 114 from the data hub 108. At this point, the needle safety shield 114, the slotted cannula 102, and the needle hub 110 may be thrown away or otherwise treated as a biohazard with appropriate disposal steps taken to treat it as such.

As described in the present disclosure, the needle safety shield 114 may have a via 203 formed through it such that the slotted cannula 102 may be passed therethrough during operation by a healthcare professional. Additionally, the needle safety shield 114, In some embodiments, may include a needle safety shield cutting blade (not shown in FIG. 2) that is used to cut the splitable sheath 104 during extraction of the slotted cannula 102 from the patient's vascular anatomy.

The data hub 108 may remain with the patient and later may be allowed to be secured to the outer surface of the patient's anatomy (e.g., the arm). As described in the present disclosure, the digital probe 106 may include a supplemental length of digital probe 120 that allows a healthcare professional to advance the digital probe 106 further into the patient's anatomy and into the vein or artery if necessary. This advancement of the digital probe 106 using the supplemental length of digital probe 120 may be accomplished while the slotted cannula 102 is within the vein or artery. Therefore, during removal of the slotted cannula 102, the length of the supplemental length of digital probe 120 may change as the healthcare professional deems is necessary or not to pass the digital probe 106 further into the patient's vein or artery.

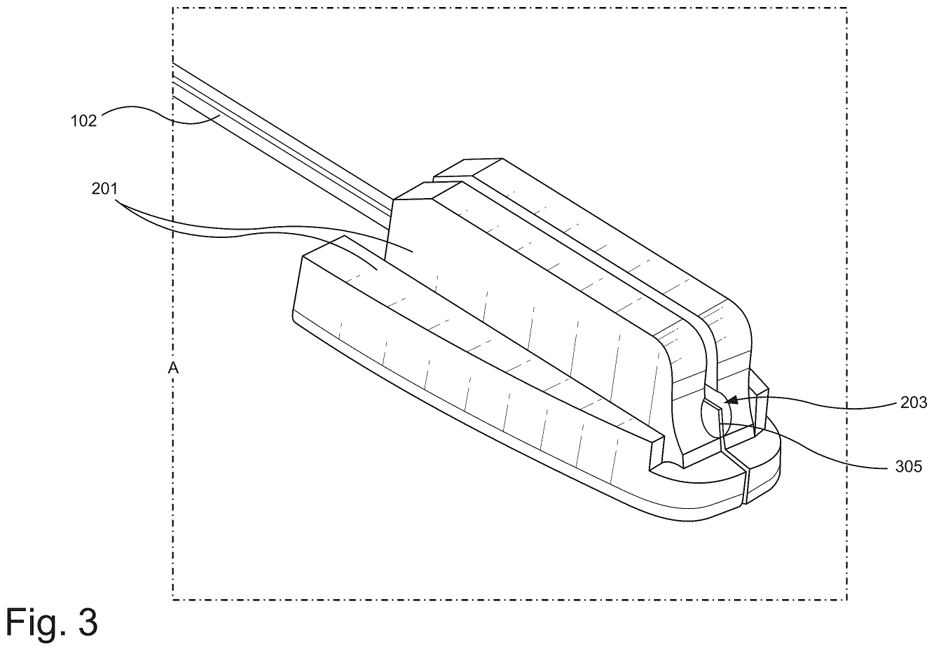

FIG. 2 includes a dashed box “A” generally encompassing the needle safety shield 114 shown in FIG. 2. Box “A” is also included in FIG. 3 to highlight and show a larger image of the needle safety shield 114 and a portion of the slotted cannula 102. FIG. 3 is a side perspective view of a needle safety shield 114 of a vascular probe insertion assembly 100 similar to the vascular probe insertion assembly 100 shown in FIGS. 1 and 2 according to some embodiments of the present disclosure. FIG. 3 shows a closer view of the needle safety shield 114 including the via 203 and a needle safety shield cutting blade 305 formed on the needle safety shield 114. FIG. 3 also shows the slotted cannula 102 in a most retracted position with a beveled portion of the slotted cannula 102 being fully retracted into the via 203.

As described in the present disclosure, the needle safety shield 114 may serve as both a shield used to protect the healthcare professional from being pricked by the used slotted cannula 102 as well as to serve to cut the splitable sheath (not shown in FIG. 3) during operation. In some embodiments, the needle safety shield cutting blade 305 may be placed at a distal end of the via 203 such that the splitable sheath may be cut by the needle safety shield cutting blade 305. The splitable sheath may be cut by the needle safety shield cutting blade 305 as the slotted cannula 102 and splitable sheath are pulled through the via 203 of the needle safety shield 114. This causes the splitable sheath to be opened along its entire length allowing the digital probe (not shown in FIG. 3) to move out of the slotted cannula 102 and being released from the remaining portions of the vascular probe insertion assembly 100 as described in the present disclosure.

In some embodiments, the needle safety shield cutting blade 305 may be placed at a bottom surface of a distal portion of the via 203 formed through the needle safety shield 114. In another embodiment, the needle safety shield cutting blade 305 may be placed at the top or at an upper location of the via 203 formed through the needle safety shield 114. In some embodiments, the needle safety shield cutting blade 305 may be placed at a location at a distal entrance of the via 203 so that the digital probe may be concurrently released from the slotted cannula 102 as the splitable sheath is cut and split by the needle safety shield cutting blade 305 as the slotted cannula 102 is pulled through the via 203. This allows the digital probe 106 to be released from the slotted cannula 102 via the slot formed along the length of the slotted cannula 102 as the splitable sheath is cut and split by the needle safety shield cutting blade 305.

Again, the needle safety shield 114 may include a number of attachment surfaces 201 that interface with complementary attachment surfaces formed on the digital probe (not shown in FIG. 3). The attachment surfaces 201 allow the needle safety shield 114 to be operatively coupled, at least temporarily, to the bottom side of the data hub 108 during insertion of the slotted cannula 102 into the vascular anatomy of the patient. As described in the present disclosure, after the slotted cannula 102 has been inserted into the patient's vein or artery, it may be subsequently removed with the digital probe and data hub remaining in place. In the embodiments in the present disclosure, the removal of the slotted cannula 102 causes the digital probe to remain within the patient's vein or artery while the data hub (operatively coupled to the digital probe) remains with the patient. By laterally sliding the needle safety shield 114 away from the data hub 108, the needle safety shield 114, slotted cannula 102, and needle hub (not shown in FIG. 3) are removed away from the data hub thereby separating the needle safety shield 114 from the data hub. At this point, the needle safety shield 114, slotted cannula 102, and needle hub may be thrown away or otherwise treated as a biohazard with appropriate disposal steps taken to treat it as such.

In some embodiments, the needle safety shield 114 may include a trench or digital probe channel 118 formed along the needle safety shield 114 that matches the trench formed in the data hub. These trenches formed through the needle safety shield 114 and the data hub allow the digital probe to be separated from the data hub so that the supplemental length of digital probe may be secured to the patient's anatomy along with the data hub.

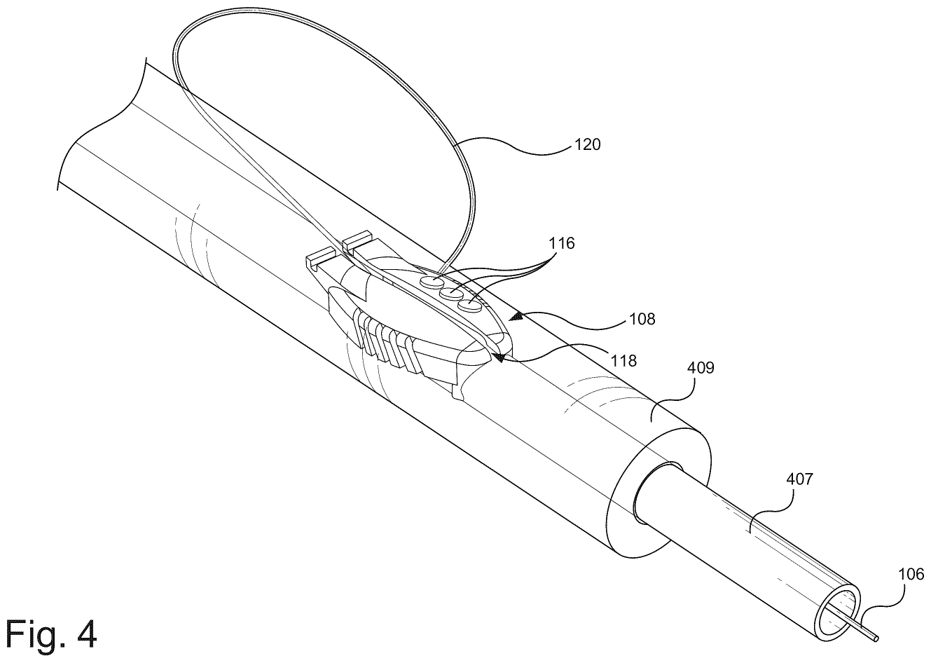

FIG. 4 is a perspective view of a digital probe 106 of a vascular probe insertion assembly 100 accessing a vascular anatomy 407 according to some embodiments of the present disclosure. FIG. 4 shows the digital probe 106 in an installed position after a healthcare professional has removed the slotted cannula, the splitable sheath, the needle safety shield, and the needle hub shown in, for example, FIGS. 1 and 2. As described in the present disclosure, as the slotted cannula is pulled out of the patient's vascular anatomy 407, the digital probe 106 remains. In some embodiments, a supplemental length of digital probe 120 may be advanced further into the patient's vascular anatomy by the healthcare professional after the slotted cannula is inserted into the vascular anatomy 407 but before the slotted cannula is removed from the patient's anatomy. This allows the advancement of the digital probe 106 further into the patient's vascular anatomy 407 prior to the slotted cannula being removed to, for example, cause the digital probe 106 to remain within the vascular anatomy and preventing the slotted cannula from dragging the digital probe 106 back out of the vascular anatomy 407. Additionally, while the slotted cannula is being pulled out of the patient's vascular anatomy 407, the digital probe 106/supplemental length of digital probe 120 may exit the slotted cannula due to the splitable sheath being cut and the slot formed in the slotted cannula serving as an exit for the digital probe 106 to be removed from the slotted cannula.

In some embodiments, the data hub 108 may be secure to the external anatomy 409 of the patient. The external anatomy 409 to which the data hub is secured may vary depending on the vascular anatomy 407 the digital probe 106 is inserted into. For example, where the vascular anatomy 407 is a vein within a patient's arm, the data hub 108 may be secured to the patient's arm close to where the digital probe 106 has passed through the patient's skin.

As described in the present disclosure, the vascular probe insertion assembly 100 may also include a data hub 108 operatively coupled to the digital probe 106. The data hub 108 may include any type of circuitry used to receive and transmit data detected by the digital probe 106 when the digital probe 106 has been successfully inserted into the patient's vein or artery. In some embodiments, this circuitry may include a wireless radio or other transmitting device that allows for the wireless transmission of the data detected at the digital probe 106 to be transmitted to, for example, a vascular monitoring system that is described in the present disclosure. Additionally, the data hub 108 may include a power source, a PMU, and a microcontroller or other hardware processing device, among other circuitry.

In some embodiments, the data hub 108 may include one or more visual indicators 116 used to signal to the healthcare professionals that, for example, the data hub 108 is detecting parameters of a patient's vascular anatomy due to the digital probe 106 being properly inserted into the patient's vascular anatomy. These visual indicators 116 may include a variety of differently colored light emitting diodes (LEDs) that may indicate proper placement of the digital probe 106 (e.g., green LED lighted), improper placement of the digital probe 106 (e.g., red LED lighted), or sub-optimal placement of the digital probe 106 (e.g., yellow LED lighted). In some embodiments, the data detected (or not) by the digital probe 106 may be used by the microcontroller in the data hub 108 to determine whether the digital probe 106 has been properly, improperly, or sub-optimally inserted into the patient's anatomy. In some embodiments, the data hub 108 may be secured to the external anatomy 409 of the patient using, for example, a transparent film dressing (e.g., 3M® TEGADERM® transparent film dressing) so that the healthcare professional can still view the visual indicators 116 present on the data hub 108. Other securing devices may be used to secure the data hub 108 to the patient's external anatomy 409. Still further, the securing devices may be selectively removable such that a healthcare professional may remove the securing devices either to swap out a new set of securing devices or to eventually remove the digital probe 106 and data hub 108 when monitoring of the patient's vascular anatomy 407 is no longer necessary.

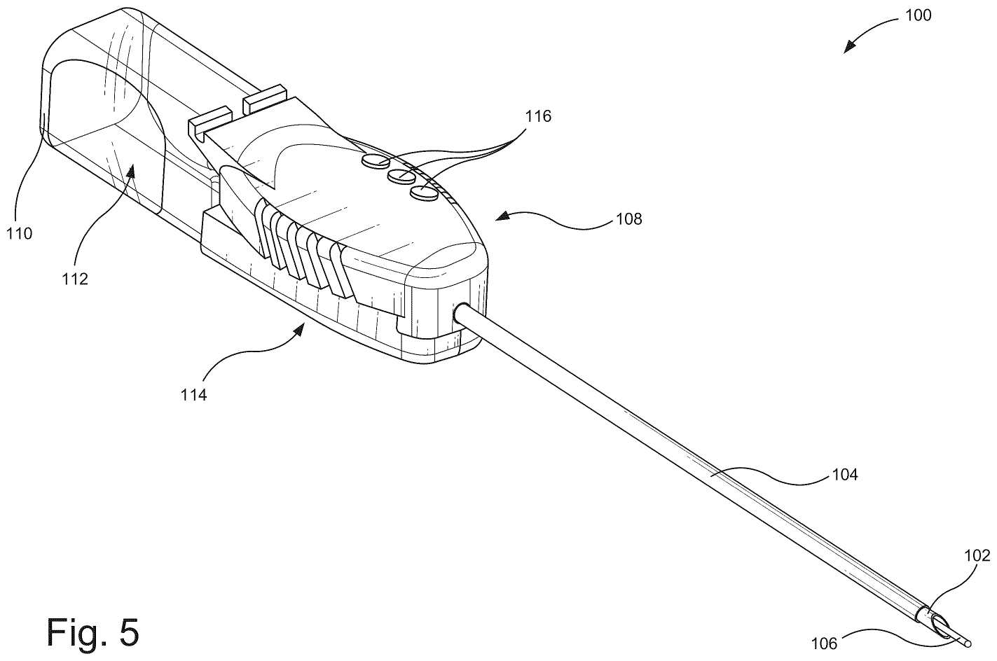

FIG. 5 is a perspective view of a vascular probe insertion assembly 100 according to some embodiments of the present disclosure. The vascular probe insertion assembly 100 shown in FIG. 5 may be similar to that described in connection with FIG. 1, for example. The vascular probe insertion assembly 100 may include a slotted cannula 102. The slotted cannula 102 may include slot formed along the length of the slotted cannula 102 into which a digital probe 106 may be placed. The slot formed in the slotted cannula 102 forms sidewalls extending along the length of the slotted cannula 102 with the sidewalls maintaining the digital probe 106 coaxially within except at the slot. The digital probe 106 may, therefore, run coaxially within the slotted cannula 102 until the slotted cannula 102 is removed from the patient's anatomy as described in the present disclosure.

The digital probe 106 may include, In some embodiments, a core wire for structure and durability during advancement of the digital probe 106 through the patient's vascular anatomy. The core wire may be a nitinol core wire that runs the length of the digital probe 106 either at the center axis of the digital probe 106 or offset from the center axis. In some embodiments, the digital probe 106 may include optical fibers and/or wires connected to one or more sensors placed along the length of the digital probe 106. In some embodiments, a distal end of the digital probe 106 may include an atraumatic tip that reduces the risk of the digital probe 106 inducing damage or complications in the patient's vein or artery. In some embodiments, a proximal end of the digital probe 106 may be operatively coupled within the digital probe 106 and, unlike the example embodiment shown in FIG. 1, the digital probe 106 does not include a supplemental length of digital probe. This allows the healthcare professional the ability to secure the data hub 108 to the external anatomy of a patient without having to also secure an unused portion of the digital probe 106 to the patient's external anatomy. Additionally, with a fixed length of the digital probe 106, the healthcare professional may understand that when the vascular probe insertion assembly 100 is appropriately used to insert the digital probe 106, the digital probe 106 is set within the vascular anatomy of the patient at a length that is necessary to complete the monitoring of the critical vascular-based parameters.

In some embodiments, the digital probe 106 may be coated with a variety of coatings used to improve the performance of the digital probe 106 as well as reduce the risk of complications such as thrombus or probe-related blood stream infections (e.g., sepsis). These coatings may include silicon lubes that may or may not include an antimicrobial additive such as alkanes or saturated hydrocarbons (e.g., CH (x)). In some embodiments, the digital probe 106 may be coated with any of anti-thrombogenic or anti-microbial coatings and/or polymer additives.

The sensors present on the digital probe 106 may include any number or type of sensors that detect or measure parameters of a patient's vascular anatomy. These sensors may be placed along the digital probe 106 as individual sensors or bundles of sensors. These sensors may include sensors that incorporate technologies that can detect or measure a patient's blood pressure (venous or arterial), blood gases, blood pH levels, and presence of electrolytes among other types of sensors. In some embodiments, the sensors present on the digital probe 106 may be selected to detect or measure other targeted physiological or procedural parameters.

The vascular probe insertion assembly 100 also may include a splitable sheath 104 formed around the slotted cannula 102. In some embodiments, the splitable sheath 104 may extend the entire length of the slotted cannula 102 except along a beveled portion of the slotted cannula 102. This allows the sharp terminal end of the slotted cannula 102 to penetrate a patient's skin when the healthcare professional accesses the patient's vein or artery. The splitable sheath 104 also prevents the digital probe 106 from exiting the slot formed along the slotted cannula 102 during insertion of the vascular probe insertion assembly 100 into the patient's vascular anatomy.

The vascular probe insertion assembly 100 may include a needle hub 110 operatively coupled to a proximal end of the slotted cannula 102 opposite the beveled portion of the slotted cannula 102 that forms a sharpened tip configured to be inserted into a patient's vascular anatomy. The needle hub 110, in an embodiment may include a hollow chamber or fluid reservoir 112 fluidically coupled to the interior of the slotted cannula 102. The fluid reservoir 112 may receive an amount of blood as the healthcare professional inserts the slotted cannula 102 into the patient's vascular anatomy. In this way, the healthcare professional may determine if and when the slotted cannula 102 has penetrated a vein or artery of the patient by viewing whether a flow of blood has entered the fluid reservoir 112. The volume of the fluid reservoir 112 may be sufficient to allow the healthcare professional to view this process while also not drawing a significant amount of blood from the patient. Because the fluid reservoir 112 is used to merely detect when the slotted cannula 102 has reached a vein or artery, the fluid reservoir 112 along with the slotted cannula 102 and needle safety shield 114 described in the present disclosure may be disposed of when the digital probe 106 has been introduced into the patient's vascular anatomy and the slotted cannula 102 has been pulled out of the vein or artery.

The vascular probe insertion assembly 100 also may include a needle safety shield 114. The needle safety shield 114 is formed around a portion of the slotted cannula 102 and is allowed to move along the shaft of the slotted cannula 102 during operation. In some embodiments, the needle safety shield 114 has a via formed through it such that the slotted cannula 102 may be passed therethrough. Additionally, the needle safety shield 114, In some embodiments, may include a needle safety shield cutting blade (not shown in FIG. 1) that is used to cut the splitable sheath 104 during extraction of the slotted cannula 102 from the patient's vascular anatomy.

The vascular probe insertion assembly 100 may include a data hub 108 operatively coupled to the digital probe 106. The data hub 108 may include any type of circuitry used to receive and transmit data detected by the digital probe 106 when the digital probe 106 has been successfully inserted into the patient's vein or artery. In some embodiments, this circuitry may include a wireless radio or other transmitting device that allows for the wireless transmission of the data detected at the digital probe 106 to be transmitted to, for example, a vascular monitoring system that is described in the present disclosure. Additionally, the data hub 108 may include a power source, a power management unit (PMU), and a microcontroller or other hardware processing device, among other circuitry. In some embodiments, the data hub 108 may include one or more visual indicators 116 used to signal to the healthcare professionals that, for example, the data hub 108 is detecting parameters of a patient's vascular anatomy due to the digital probe 106 being properly inserted into the patient's vascular anatomy. These visual indicators 116 may include a variety of differently colored light emitting diodes (LEDs) that may indicate proper placement of the digital probe 106 (e.g., green LED lighted), improper placement of the digital probe 106 (e.g., red LED lighted), or sub-optimal placement of the digital probe 106 (e.g., yellow LED lighted). In some embodiments, the data detected (or not) by the digital probe 106 may be used by the microcontroller in the data hub 108 to determine whether the digital probe 106 has been properly, improperly, or sub-optimally inserted into the patient's anatomy.

During operation of the vascular probe insertion assembly 100, the healthcare professional may address the patient's anatomy and determine a location where the digital probe 106 may be placed within the patient's vein or artery. Once an appropriate injection site has been determined, the healthcare professional may insert the vascular probe insertion assembly 100 and, specifically, the slotted cannula 102 with its digital probe 106 and the splitable sheath 104 into the patient's anatomy in into a vein or artery. At this point, because the slotted cannula 102 is fluidically coupled with the fluid reservoir 112 of the needle hub 110, the healthcare professional may visually detect that the slotted cannula 102 has reached a vein or artery by the presence of blood within the fluid reservoir 112. Because the needle hub 110 is made of a clear plastic, for example, the healthcare professional may visually detect the filling of the fluid reservoir 112.

Once the healthcare professional has detected the presence of blood in the fluid reservoir 112 of the needle hub 110 or visualized blood flashback indicating that the slotted cannula 102 has reached a vein or artery, the healthcare professional may advance the digital probe 106 distally into the vasculature to ensure access to the vasculature is not lost during removal or proximal withdrawal of the slotted cannula 102. In some embodiments, after the digital probe 106 is advanced distally with respect to the slotted cannula 102, the healthcare professional may withdraw the slotted cannula 102. In some embodiments, the healthcare professional may do this by grasping the needle hub 110 in one hand and the data hub 108/needle safety shield 114 with the other. While holding the data hub 108/needle safety shield 114 in place, the healthcare professional may begin to pull the needle hub 110 away from the patient and the remaining portions of the vascular probe insertion assembly 100. By doing this, the needle safety shield cutting blade (not shown in FIG. 1) of the needle safety shield 114 may begin to cut the splitable sheath 104 as the slotted cannula 102 is pulled through the via formed through the needle safety shield 114. This will continue until the beveled portion of the slotted cannula 102 is safely housed within the via formed through the needle safety shield 114. This process also may include the removal of the data hub 108 from the needle safety shield 114. In some embodiments, the data hub 108 and needle safety shield 114 may be operatively coupled together via a rail system such that they may be slidably removed from each other as the healthcare professional pulls the slotted cannula 102 and splitable sheath 104 through the via formed through the needle safety shield 114.

As the beveled end of the slotted cannula 102 enters the via formed through the needle safety shield 114, the slotted cannula 102 may be prevented from totally being removed from the via in order to prevent the sharp point of the slotted cannula 102 from touching the healthcare professional and potentially harming the healthcare professional. Additionally, the splitable sheath 104, having been cut lengthwise along the length of the slotted cannula 102 may be separated from the slotted cannula 102 and thrown away with the needle hub 110, slotted cannula 102, and needle safety shield 114.

Additionally, as the slotted cannula 102 is pulled out of the patient's vascular anatomy, the digital probe 106 remains. In some embodiments, a supplemental length of digital probe 120 may be advanced further into the patient's vascular anatomy by the healthcare professional as the slotted cannula 102 is inserted into the vascular anatomy. This allows the advancement of the digital probe 106 further into the patient's vascular anatomy prior to the slotted cannula 102 being removed to, for example, cause the digital probe 106 to remain within the vascular anatomy and preventing the slotted cannula 102 from dragging the digital probe 106 back out of the vascular anatomy. Additionally, while the slotted cannula 102 is being pulled out of the patient's vascular anatomy, the digital probe 106/supplemental length of digital probe 120 may exit the slotted cannula 102 due to the splitable sheath 104 being cut and the slot formed in the slotted cannula 102 serving as an exit for the digital probe 106 to be removed from the slotted cannula 102.

When the slotted cannula 102, the splitable sheath 104, the needle safety shield 114, and the needle hub 110 have been removed from the digital probe 106 and the data hub 108, the healthcare professional may determine whether the digital probe 106 has been successfully inserted into the patient's vascular anatomy. This may be done by determining visually whether the digital probe 106 appears to be passing into the patient's anatomy and whether the visual indicators 116 on the data hub 108 indicate proper insertion of the digital probe 106. Where the healthcare professional has determined improper insertion of the digital probe 106, the digital probe 106 may be removed by pulling the digital probe 106 from the patient's vascular anatomy and a new insertion site may be determined using a new vascular probe insertion assembly 100. Where the healthcare professional has determined that the digital probe 106 is properly inserted, the healthcare professional may secure the data hub 108 to the exterior portion of the patient's anatomy (e.g., arm) and apply a securement dressing (not shown in FIG. 1) over the data hub 108/supplemental length of digital probe 120 to prevent dislodgement of the digital probe 106. In some embodiments, the healthcare professional may also apply a skin adhesive at the injection site where the slotted cannula 102 entered the patient's body to secure the digital probe 106 to the patient during indwelling of the digital probe 106.

Where the digital probe 106 and data hub 108 are no longer needed by the healthcare professional to detect or measure parameters of a patient's vascular anatomy, the healthcare professional may remove the digital probe 106 and data hub 108 and throw them away. In some embodiments, the healthcare professional may do this by gripping the data hub 108 and pulling on the coupled digital probe 106 away from the patient's body. This allows the digital probe 106 to be pulled out of the vein or artery, through the remaining portions of the patient's anatomy, and out of the hole formed by the slotted cannula 102 during insertion.

The vascular probe insertion assembly 100 described in the present disclosure provides for direct in-vascular measure of hemodynamic and blood based critical patient parameters with a minimum footprint on the patient's body. The vascular probe insertion assembly 100 also allows for continuous or intermittent monitoring without the procedural and workflow difficulties associated with other monitoring systems. Further, the use of the vascular probe insertion assembly 100 described in the present disclosure does not require use of a separate catheter or vascular access device. Still further, risks associated with other systems used to complete the measurements that the vascular probe insertion assembly 100 can accomplish. The vascular probe insertion assembly 100 described in the present disclosure may be used for short term or long-term monitoring of critical vascular-based parameters while the digital probe 106 is in place.

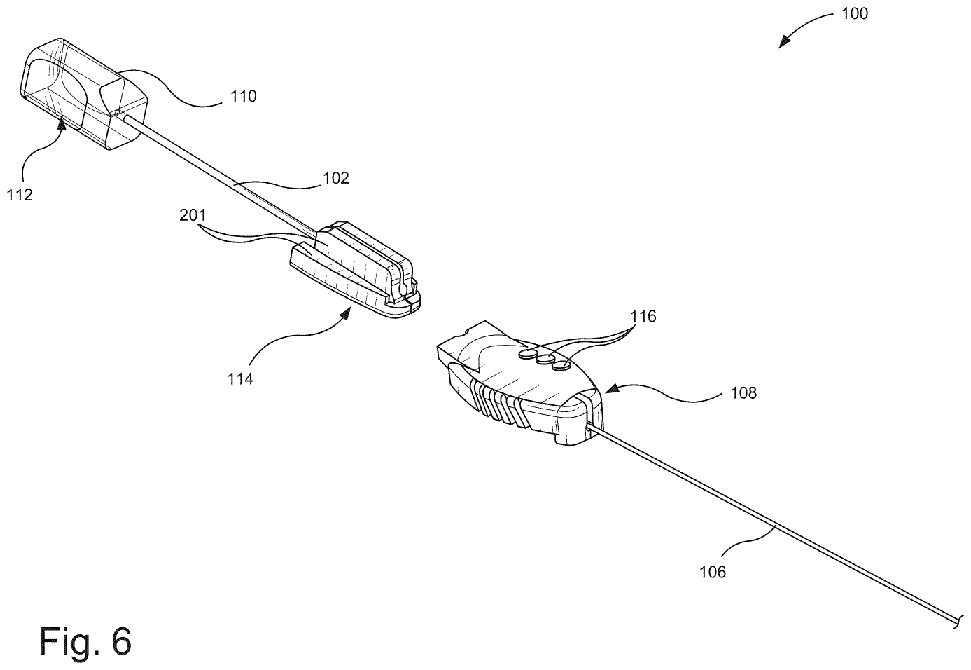

FIG. 6 is a side perspective view of a vascular probe insertion assembly 100 according to some embodiments of the present disclosure. The vascular probe insertion assembly 100 shown in FIG. 6 may be similar to the vascular probe insertion assembly 100 shown in FIG. 5 with the fixed length of the digital probe 106. In the embodiment shown in FIG. 6, the digital probe 106 and data hub 108 have been removed away from the slotted cannula 102, needle safety shield 114 and needle hub 110. The splitable sheath 104 shown in FIG. 5 is not shown in FIG. 6 because, as described in the present disclosure, the movement of the needle safety shield 114 towards a distal end of the slotted cannula 102 where the bevel is formed causes a needle safety shield cutting blade of the needle safety shield 114 to cut the splitable sheath 104 away exposing the slotted cannula 102.

As depicted in FIG. 6, the needle safety shield 114 may include a number of attachment surfaces 201 that interface with complementary attachment surfaces formed on the digital probe 106. The attachment surfaces 201 allow the needle safety shield 114 to be operatively coupled, at least temporarily, to the bottom side of the data hub 108 during insertion of the slotted cannula 102 into the vascular anatomy of the patient. As described in the present disclosure, after the slotted cannula 102 has been inserted into the patient's vein or artery, it may be subsequently removed with the digital probe 106 and data hub 108 remaining in place. In the embodiments in the present disclosure, the removal of the slotted cannula 102 causes the digital probe 106 to remain within the patient's vein or artery while the data hub 108 (operatively coupled to the digital probe 106) remains with the patient. By laterally sliding the needle safety shield 114 away from the data hub 108, the needle safety shield 114, the slotted cannula 102, and the needle hub 110 are removed away from the data hub 108 thereby separating the needle safety shield 114 from the data hub 108. At this point, the needle safety shield 114, the slotted cannula 102, and the needle hub 110 may be thrown away or otherwise treated as a biohazard with appropriate disposal steps taken to treat it as such.

The data hub 108 may remain with the patient and later may be allowed to be secured to the outer surface of the patient's anatomy (e.g., arm). As described in the present disclosure, the digital probe 106 may include a supplemental length of digital probe 120 that allows a healthcare professional to advance the digital probe 106 further into the patient's anatomy and into the vein or artery if necessary. This advancement of the digital probe 106 using the supplemental length of digital probe 120 may be accomplished while the slotted cannula 102 is within the vein or artery. Therefore, during removal of the slotted cannula 102, the length of the supplemental length of digital probe 120 may change as the healthcare professional deems is necessary or not to pass the digital probe 106 further into the patient's vein or artery.

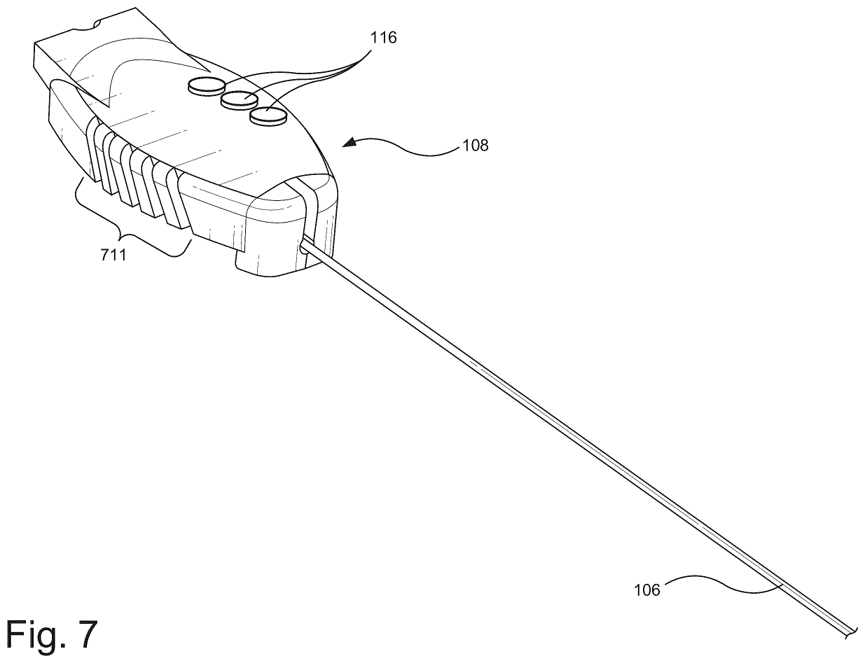

FIG. 7 is a side elevation view of a data hub 108 with its digital probe 106 according to some embodiments of the present disclosure. The data hub 108 and digital probe 106 shown in FIG. 7 may be part of the vascular probe insertion assembly 100 shown in FIG. 6 in an embodiment.

As described in the present disclosure, a proximal end of the digital probe 106 may be operatively coupled within the digital probe 106 and, unlike the example embodiment shown in FIG. 1, for example, the digital probe 106 does not include a supplemental length of digital probe. This allows the healthcare professional the ability to secure the data hub 108 to the external anatomy of a patient without having to also secure an unused portion of the digital probe 106 to the patient's external anatomy. Additionally, with a fixed length of the digital probe 106, the healthcare professional may understand that when the vascular probe insertion assembly 100 is appropriately used to insert the digital probe 106, the digital probe 106 is set within the vascular anatomy of the patient at a length that is necessary to complete the monitoring of the critical vascular-based parameters.

FIG. 7 also shows a knurled surface 711 that may be formed onto an outer surface of the data hub 108. This knurled surface 711 may include any type of surfacing that can be used by a healthcare professional to better grip the data hub 108 and the needle safety shield (not shown in FIG. 7) operatively coupled to the data hub 108 during insertion of the slotted cannula 102 into the patient's vascular anatomy 407.

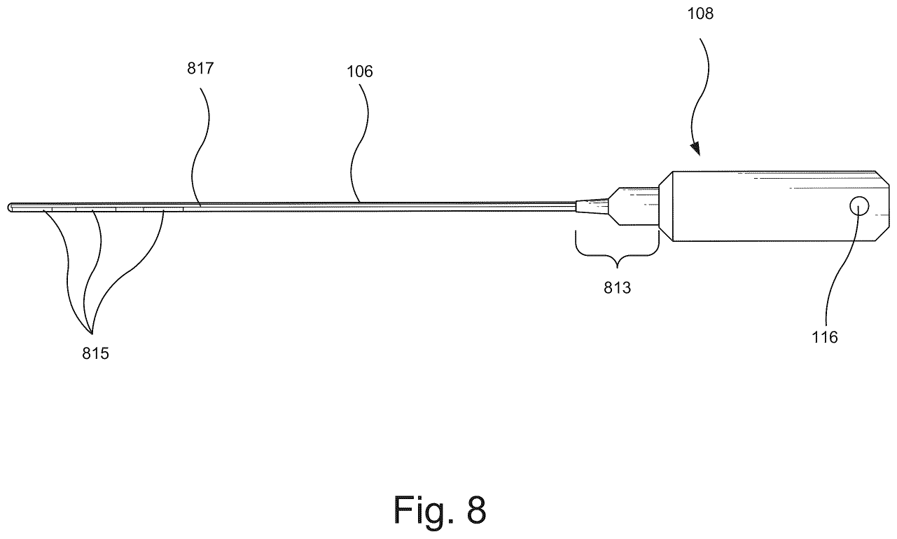

FIG. 8 is a top view of a data hub 108 and digital probe 106 according to some embodiments of the present disclosure. FIG. 8 shows other features of a data hub 108 and digital probe 106 that may be included with the vascular probe insertion assembly 100 described in the present disclosure.

The data hub 108 may include a strain relief 813 portion that operatively couples the digital probe 106 to the data hub 108. The strain relief 813 may serve to prevent damage to the digital probe 106 if and when the data hub 108 is moved relative to the digital probe 106. In some embodiments, the strain relief 813 may be used to prevent dislodgement of the digital probe 106 from the data hub 108 due to the stress caused by bending, twisting, or pulling of the data hub 108 against the digital probe 106 when the healthcare professional is attempting to secure the data hub 108 to the patient's body. In some embodiments, the strain relief 813 may be sized to help distribute the stress between the data hub 108 and digital probe 106 sufficiently to prevent damage. This will prevent the nitinol core wire 817 and other electrical connections within the digital probe 106, for example, from being damaged.

The digital probe 106 also may include one or more sensors 815. As described in the present disclosure, these sensors 815 may be any type of sensor that can detect, monitor, or sense critical vascular-based parameters within the vascular anatomy of a patient. These sensors 815 may include those sensors associated with detecting, monitoring, or sensing venous or arterial blood pressure, temperature, potential of hydrogen (pH) of the patient's blood, presence of organic compounds such as lactate, the presence and amount of oxygen (O₂) in the blood, the percentage of oxygen (SPO₂), as well as other intervascular parameters that may be detected. In some embodiments, each sensor may be operatively coupled to the data hub 108 via an electrical connection that operatively couples the sensor 815 to, for example, a microcontroller and other circuitry within the data hub 108.

In some embodiments, the data hub 108 may include one or more visual indicators 116 used to signal to the healthcare professionals that, for example, the data hub 108 is detecting parameters of a patient's vascular anatomy due to the digital probe 106 being properly inserted into the patient's vascular anatomy. These visual indicators 116 may include a variety of differently colored light emitting diodes (LEDs) that may indicate proper placement of the digital probe 106 (e.g., green LED lighted), improper placement of the digital probe 106 (e.g., red LED lighted), or sub-optimal placement of the digital probe 106 (e.g., yellow LED lighted). In some embodiments, the data detected (or not) by the digital probe 106 may be used by the microcontroller in the data hub 108 to determine whether the digital probe 106 has been properly, improperly, or sub-optimally inserted into the patient's anatomy.

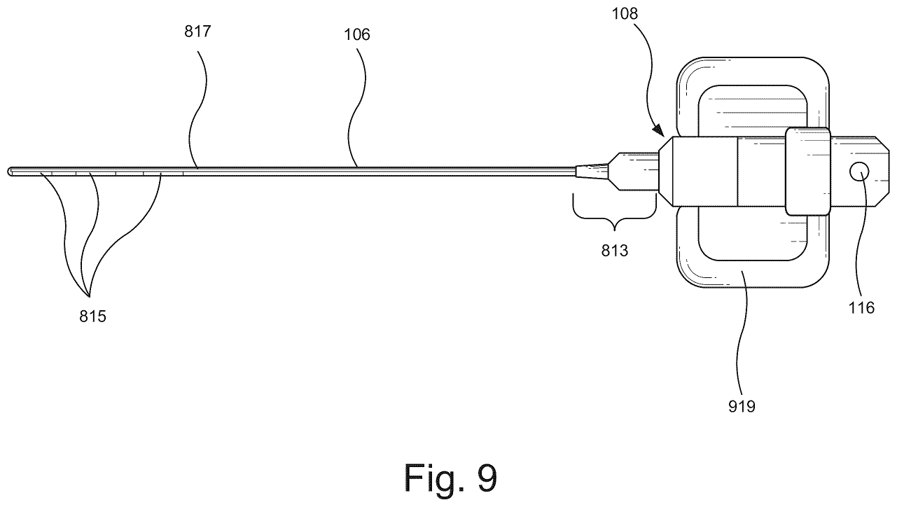

FIG. 9 is a top view of a digital probe 106 and data hub 108 according to some embodiments of the present disclosure. FIG. 9 shows a stabilization platform 919 operatively coupled to the data hub 108. The stabilization platform 919 may, in some embodiments, be semi rigid that allows the data hub 108 to be placed against the patient's external anatomy so that it does not rotate while the digital probe 106 is placed within the patient's vascular anatomy as described in the present disclosure. In some embodiments, the stabilization platform 919 may include an adhesive formed on a surface that is to abut the patient's external anatomy. In some embodiments, the stabilization platform 919 may include a non-slip surface that creates more friction between the stabilization platform 919 and the patient's external anatomy. In some embodiments, the stabilization platform 919 may reinforce the stability of the data hub 108 being placed against the patient's external anatomy.

Again, in some embodiments, the data hub 108 may include one or more visual indicators 116 used to signal to the healthcare professionals that, for example, the data hub 108 is detecting parameters of a patient's vascular anatomy due to the digital probe 106 being properly inserted into the patient's vascular anatomy. These visual indicators 116 may include a variety of differently colored light emitting diodes (LEDs) that may indicate proper placement of the digital probe 106 (e.g., green LED lighted), improper placement of the digital probe 106 (e.g., red LED lighted), or sub-optimal placement of the digital probe 106 (e.g., yellow LED lighted). In some embodiments, the data detected (or not) by the digital probe 106 may be used by the microcontroller in the data hub 108 to determine whether the digital probe 106 has been properly, improperly, or sub-optimally inserted into the patient's anatomy.

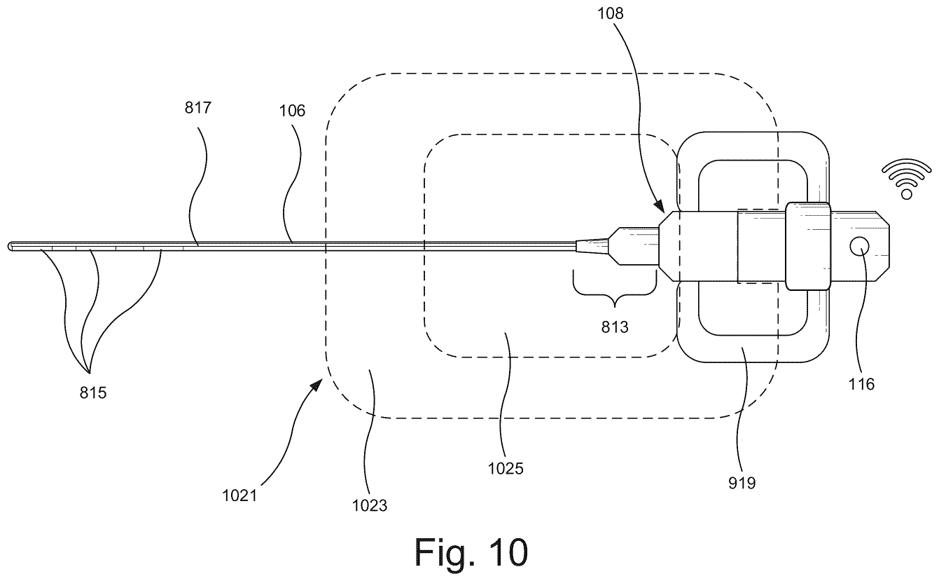

FIG. 10 is a top view of a top view of a data hub 108 and digital probe 106 according to some embodiments of the present disclosure. Again, the data hub 108 may include a stabilization platform 919 similar to that described in connection with FIG. 9.

In an embodiment shown in FIG. 10, the stabilization platform 919 is also in place. As described in the present disclosure, the stabilization platform 919 may, in some embodiments, be semi rigid that allows the data hub 108 to be placed against the patient's external anatomy so that it does not rotate while the digital probe 106 is placed within the patient's vascular anatomy as described in the present disclosure. In some embodiments, the stabilization platform 919 may include an adhesive formed on a surface that is to abut the patient's external anatomy. In some embodiments, the stabilization platform 919 may include a non-slip surface that creates more friction between the stabilization platform 919 and the patient's external anatomy. In some embodiments, the stabilization platform 919 may reinforce the stability of the data hub 108 being placed against the patient's external anatomy.

Additionally, during operation, the healthcare professional may secure the data hub 108 to the patient's external anatomy using a securement dressing 1021. In some embodiments, the securement dressing 1021 may include a rigid border 1023 that may include an adhesive used to tightly secure the securement dressing 1021 to the patient's external anatomy 409. In some embodiments, the securement dressing 1021 may sandwich the data hub 108 between a bottom surface of the securement dressing 1021 and the surface of the patient's external anatomy. In some embodiments, the securement dressing 1021 also may include a transparent window 1025 that allows the healthcare professional to view the data hub 108 as the digital probe 106 is within the patient's vascular anatomy. In some embodiments, the transparent window 1025 allows for the healthcare professional to determine if the digital probe 106 is being maintained within the patient's vascular anatomy 407 as well as the status of the data hub 108. As described in the present disclosure, the data hub 108 may include one or more visual indicators 116 used to signal to the healthcare professionals that, for example, the data hub 108 is detecting parameters of a patient's vascular anatomy due to the digital probe 106 being properly inserted into the patient's vascular anatomy. This transparent window 1025, therefore, allows the healthcare professional to view the status of the visual indicators 116. In some embodiments, the securement dressing 1021 may be 3M® TEGADERM® transparent film dressing.

Again, in some embodiments, the data hub 108 may include one or more visual indicators 116 used to signal to the healthcare professionals that, for example, the data hub 108 is detecting parameters of a patient's vascular anatomy due to the digital probe 106 being properly inserted into the patient's vascular anatomy. These visual indicators 116 may include a variety of differently colored light emitting diodes (LEDs) that may indicate proper placement of the digital probe 106 (e.g., green LED lighted), improper placement of the digital probe 106 (e.g., red LED lighted), or sub-optimal placement of the digital probe 106 (e.g., yellow LED lighted). In some embodiments, the data detected (or not) by the digital probe 106 may be used by the microcontroller in the data hub 108 to determine whether the digital probe 106 has been properly, improperly, or sub-optimally inserted into the patient's anatomy.

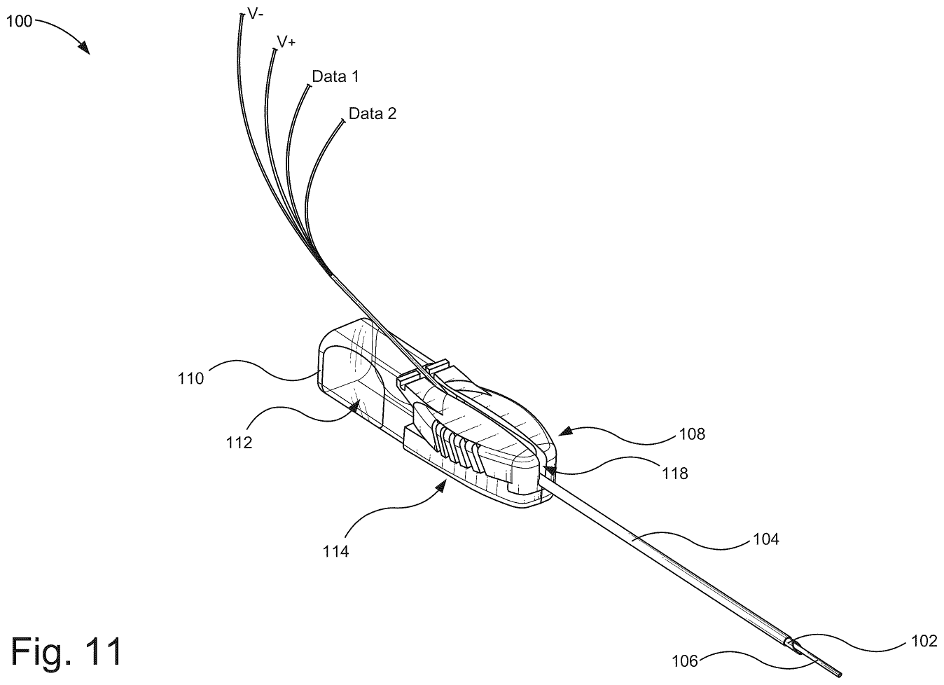

FIG. 11 is a side perspective view of a vascular probe insertion assembly 100 according to some embodiments of the present disclosure. As described in the present disclosure, the vascular probe insertion assembly 100 may include the slotted cannula 102, the splitable sheath 104, and the digital probe 106 as described in the present disclosure. The example embodiment, shown in FIG. 11 shows the digital probe 106 extending through the slotted cannula 102 and splitable sheath 104, through the digital probe channel 118 and extending out of the digital probe channel 118. FIG. 11 also shows various electrical lines within the digital probe 106 that may be used to operatively couple the digital probe 106 to, for example, monitoring system as described in the present disclosure.

In the embodiment shown in FIG. 11, the sharp end of the slotted cannula 102 may be inserted into the patient's vascular anatomy 407 so as to introduce the digital probe 106 into a vein or artery as described in the present disclosure. Additionally, when the patient's vascular anatomy 407 has been accessed and the digital probe 106 is put into place, the healthcare professional may hold the needle hub 110 with one hand and the data hub 108 in another and continue to hold the data hub 108 while the healthcare professional pulls the needle hub 110 away from the data hub 108. As this occurs, the slotted cannula 102 and splitable sheath 104 is pulled through a via formed in the needle safety shield 114. As the splitable sheath 104 is pulled through the via of the needle safety shield 114, a needle safety shield cutting blade (not shown in FIG. 11) cuts the splitable sheath 104 open allowing a length of the digital probe 106 to exit from the slotted cannula 102 as described in the present disclosure. In the embodiment shown in FIG. 11, the digital probe 106 is not operatively coupled to the data hub 108 and instead, the data hub 108 may be disposed of along with the slotted cannula 102, splitable sheath 104, and needle hub 110. This creates a situation where only the digital probe 106 is present with the patient after the digital probe 106 has been successfully inserted into the patient's vascular anatomy.

As shown in FIG. 11, the digital probe 106 may include a plurality of electrical wires that allow the sensors of the digital probe 106 to be operatively coupled to a monitoring system as described in the present disclosure. The number of electrical wires may include, for example, a negative voltage rail (V−), a positive voltage rail (V+), and any number of data electrical lines (e.g., Data 1, Data 2, etc.). The negative voltage rail and positive voltage rail may be provided to supply a current to the sensors of the digital probe 106 so that the sensors may detect, monitor, or otherwise sense the critical vascular-based parameters described in the present disclosure. Each data line may be used to pass data from the sensors to the monitoring system according to the example embodiments in the present disclosure. It is appreciated that although FIG. 11 shows only two data line (e.g., Data 1 and Data 2), the digital probe 106 may include more than or less than these two data lines.

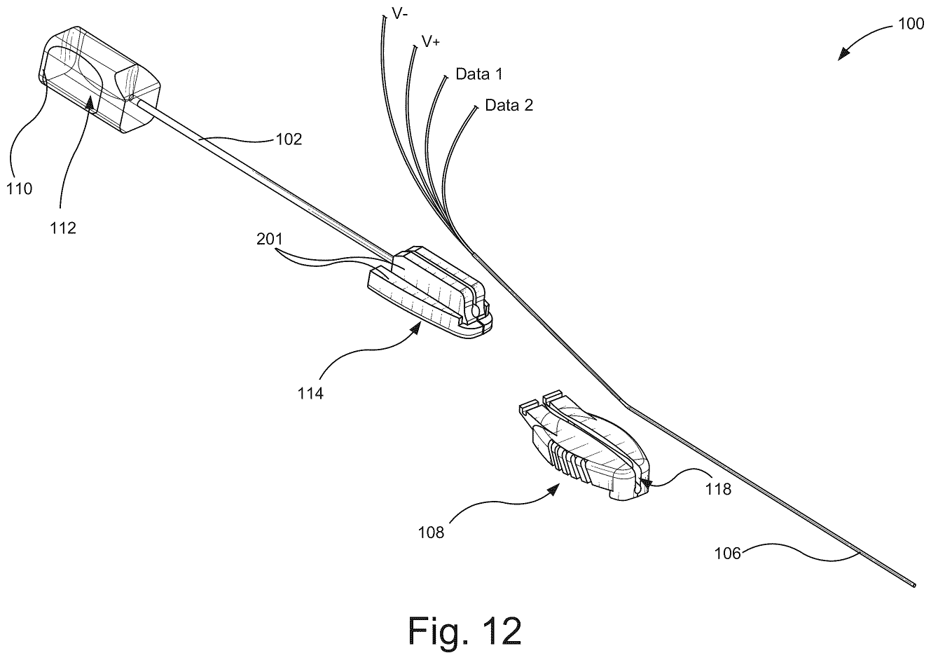

FIG. 12 is a side perspective view of a vascular probe insertion assembly 100 according to some embodiments of the present disclosure. The vascular probe insertion assembly 100 is shown in an orientation where the digital probe 106 has been placed within the patient's vascular anatomy. As described in the present disclosure, the retraction of the slotted cannula 102 from within the patient's vascular anatomy, causes the splitable sheath 104 to be cut by the needle safety shield cutting blade of the needle safety shield 114 causing the splitable sheath 104 to fall away from the vascular probe insertion assembly 100. The needle safety shield 114 then covers a sharp point of the slotted cannula 102 such that a healthcare professional is not injured by this sharp point. Additionally, in FIGS. 11, 12, and 13, the data hub 108 is not maintained with the patient and is also disposed of with the slotted cannula 102, splitable sheath 104, needle hub 110 (with its fluid reservoir 112) and needle safety shield 114. Again, this leaves the digital probe 106 only with the patient. The digital probe 106 may then be coupled to a monitoring system as described in the present disclosure.

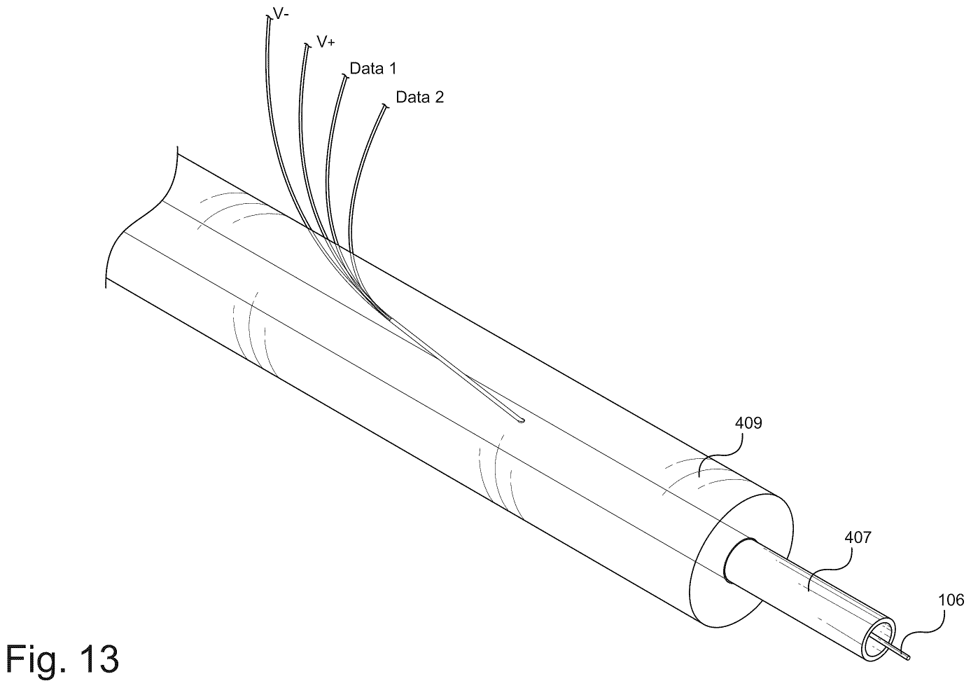

FIG. 13 is a side perspective view of a digital probe 106 inserted into vascular anatomy 407 according to some embodiments of the present disclosure. FIG. 13 shows the digital probe 106 in an installed position after a healthcare professional has removed the slotted cannula, splitable sheath, needle safety shield, data hub, and needle hub shown in, for example, FIG. 11. As described in the present disclosure, as the slotted cannula is pulled out of the patient's vascular anatomy 407, the digital probe 106 remains. The external anatomy 409 to which a portion of the digital probe 106 is secured may vary depending on the vascular anatomy 407 the digital probe 106 is inserted into. For example, where the vascular anatomy 407 is a vein within a patient's arm, the portion of the digital probe 106 may be secured to the patient's arm close to where the digital probe 106 has passed through the patient's skin and vascular anatomy 407.

Again, the digital probe 106 may include a plurality of electrical wires that allow the sensors of the digital probe 106 to be operatively coupled to a monitoring system as described in the present disclosure. The number of electrical wires may include, for example, a negative voltage rail (V−), a positive voltage rail (V+), and any number of data electrical lines (e.g., Data 1, Data 2, etc.). The negative voltage rail and positive voltage rail may be provided to supply a current to the sensors of the digital probe 106 so that the sensors may detect, monitor, or otherwise sense the critical vascular-based parameters described in the present disclosure. Each data line may be used to pass data from the sensors to the monitoring system according to the example embodiments in the present disclosure. It is appreciated that although FIG. 11 shows only two data line (e.g., Data 1 and Data 2), the digital probe 106 may include more than or less than these two data lines.

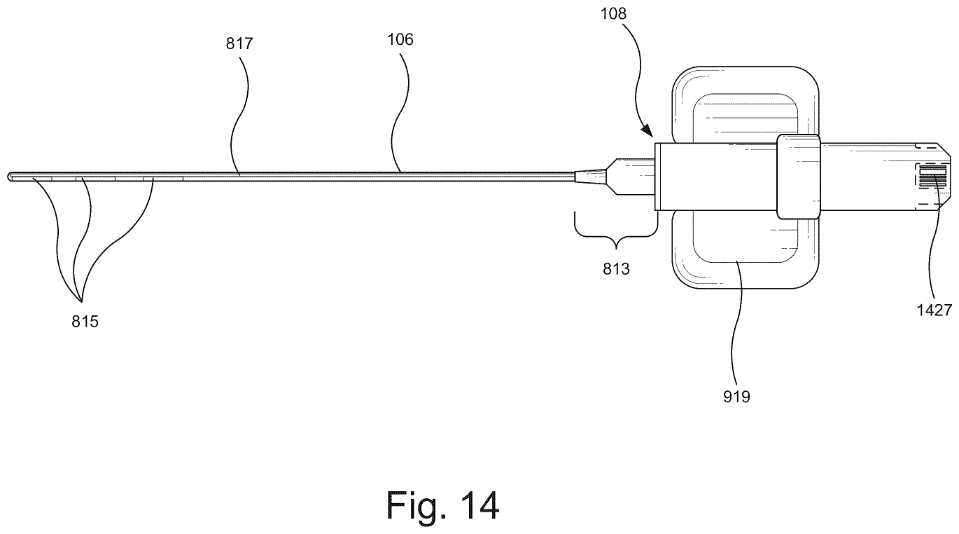

FIG. 14 is a top view of a digital probe 106 and data hub 108 according to some embodiments of the present disclosure. Similar to FIG. 9, FIG. 14 shows a stabilization platform 919 operatively coupled to the data hub 108. The stabilization platform 919 may, In some embodiments, be semi rigid that allows the data hub 108 to be placed against the patient's external anatomy so that it does not rotate while the digital probe 106 is placed within the patient's vascular anatomy as described in the present disclosure. In some embodiments, the stabilization platform 919 may include an adhesive formed on a surface that is to abut the patient's external anatomy. In some embodiments, the stabilization platform 919 may include a non-slip surface that creates more friction between the stabilization platform 919 and the patient's external anatomy. In some embodiments, the stabilization platform 919 may reinforce the stability of the data hub 108 being placed against the patient's external anatomy.

Additionally, in the embodiment shown in FIG. 14, the data hub 108 may include a strain relief 813 portion that operatively couples the digital probe 106 to the data hub 108. The strain relief 813 may serve to prevent damage to the digital probe 106 if and when the data hub 108 is moved relative to the digital probe 106. In some embodiments, the strain relief 813 may be used to prevent dislodgement of the digital probe 106 from the data hub 108 due to the stress caused by bending, twisting, or pulling of the data hub 108 against the digital probe 106 when the healthcare professional is attempting to secure the data hub 108 to the patient's body. In some embodiments, the strain relief 813 may be sized to help distribute the stress between the data hub 108 and digital probe 106 sufficiently to prevent damage. This will prevent the nitinol core wire 817 and other electrical connections within the digital probe 106, for example, from being damaged.

In some embodiments, the digital probe 106 also may include one or more sensors 815. As described in the present disclosure, these sensors 815 may be any type of sensor that can detect, monitor, or sense critical vascular-based parameters within the vascular anatomy of a patient. These sensors 815 may include those sensors associated with detecting, monitoring, or sensing venous or arterial blood pressure, temperature, potential of hydrogen (pH) of the patient's blood, presence of organic compounds such as lactate, the presence and amount of oxygen (O₂) in the blood, the percentage of oxygen (SPO₂), as well as other intervascular parameters that may be detected. In some embodiments, each sensor may be operatively coupled to the data hub 108 via an electrical connection that operatively couples the sensor 815 to, for example, a microcontroller and other circuitry within the data hub 108.

In some embodiments, the data hub 108 may include an electrical interface 1427. The electrical interface 1427 may be used by the data hub 108 to couple the data hub 108 to a monitoring system via a wired connection. In some embodiments, the electrical interface 1427 may include one or more contact pins that interface with a corresponding wired connection that operatively couples the data hub 108 to the monitoring system. As described in the present disclosure, the data hub 108 may be a wireless data hub 108 or a wired data hub 108 and the example shown in FIG. 14 is a wired data hub 108 that allows a healthcare professional to connect a wired connection interface to the proximal end of the data hub 108.

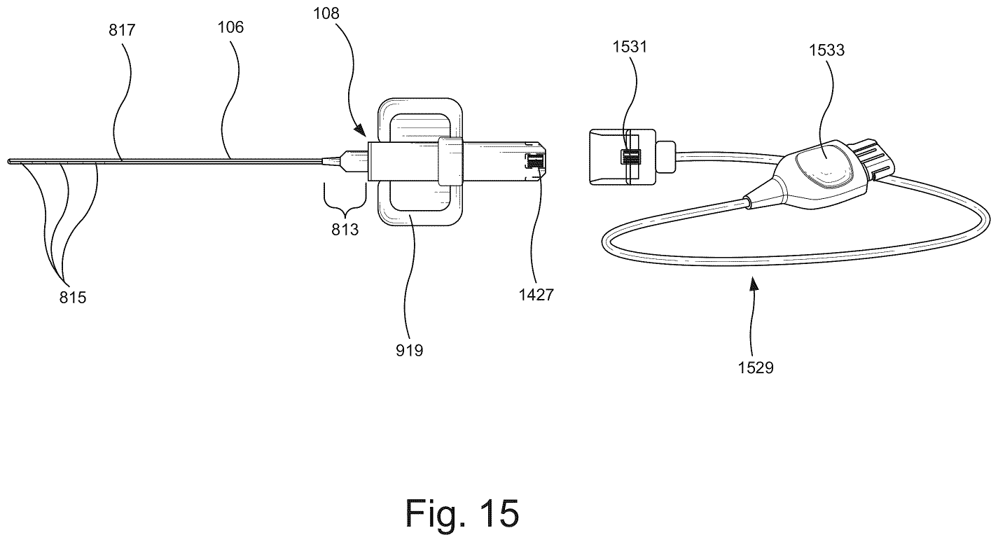

FIG. 15 is a top view of a digital probe 106, data hub 108, and a wired connection 1529 according to some embodiments of the present disclosure. FIG. 15, again, shows a stabilization platform 919 operatively coupled to the data hub 108. The stabilization platform 919 may, In some embodiments, be semi rigid that allows the data hub 108 to be placed against the patient's external anatomy so that it does not rotate while the digital probe 106 is placed within the patient's vascular anatomy as described in the present disclosure. In some embodiments, the stabilization platform 919 may include an adhesive formed on a surface that is to abut the patient's external anatomy. In some embodiments, the stabilization platform 919 may include a non-slip surface that creates more friction between the stabilization platform 919 and the patient's external anatomy. In some embodiments, the stabilization platform 919 may reinforce the stability of the data hub 108 being placed against the patient's external anatomy.

Additionally, in the embodiment shown in FIG. 14, the data hub 108 may include a strain relief 813 portion that operatively couples the digital probe 106 to the data hub 108. The strain relief 813 may serve to prevent damage to the digital probe 106 if and when the data hub 108 is moved relative to the digital probe 106. In some embodiments, the strain relief 813 may be used to prevent dislodgement of the digital probe 106 from the data hub 108 due to the stress caused by bending, twisting, or pulling of the data hub 108 against the digital probe 106 when the healthcare professional is attempting to secure the data hub 108 to the patient's body. In some embodiments, the strain relief 813 may be sized to help distribute the stress between the data hub 108 and digital probe 106 sufficiently to prevent damage. This will prevent the nitinol core wire 817 and other electrical connections within the digital probe 106, for example, from being damaged.

In some embodiments, the digital probe 106 also may include one or more sensors 815. As described in the present disclosure, these sensors 815 may be any type of sensor that can detect, monitor, or sense critical vascular-based parameters within the vascular anatomy of a patient. These sensors 815 may include those sensors associated with detecting, monitoring, or sensing venous or arterial blood pressure, temperature, potential of hydrogen (pH) of the patient's blood, presence of organic compounds such as lactate, the presence and amount of oxygen (O₂) in the blood, the percentage of oxygen (SPO₂), as well as other intervascular parameters that may be detected. In some embodiments, each sensor may be operatively coupled to the data hub 108 via an electrical connection that operatively couples the sensor 815 to, for example, a microcontroller and other circuitry within the data hub 108.

As described in the present disclosure, the data hub 108 may include an electrical interface 1427. The electrical interface 1427 may be used by the data hub 108 to couple the data hub 108 to a monitoring system via a wired connection. In some embodiments, the electrical interface 1427 may include one or more contact pins that interface with a corresponding wired connection that operatively couples the data hub 108 to the monitoring system. As described in the present disclosure, the data hub 108 may be a wireless data hub 108 or a wired data hub 108 and the example shown in FIG. 14 is a wired data hub 108 that allows a healthcare professional to connect a wired connection interface to the proximal end of the data hub 108. The use of the electrical interface 1427 allows the monitoring system to be disconnected and reconnected when necessary to detect the blood based critical patient parameters. Connection at the digital probe 106 allows for this disconnection for improved mobility of necessary patient removal, for example.

FIG. 15 also shows a wired connection 1529 used to operatively couple the data hub 108 of the vascular probe insertion assembly 100 to the monitoring system described in the present disclosure. The wired connection 1529 may include a wired connection interface 1531 that is used to interface with the electrical interface 1427 of the data hub 108. Accordingly, the wired connection interface 1531 may include one or more interfacing contact pins that interface with the contact pins of the electrical interface 1427 on the data hub 108 in an embodiment. The wired connection 1529 also may include a plug 1533 used to complete the connection between the data hub 108 and wired connection 1529 to a monitoring system. In some embodiments, the monitoring system may include a bedside monitoring system, a computer or computing device, a data cloud or a combination thereof. In some embodiments, the monitoring system may be used to compute, classify, process, transmit, receive, retrieve, originate, switch, store, display, manifest, detect, record, reproduce, handle, or use any form of information, intelligence, or data for, in an example embodiment, medical diagnosis or other purposes.

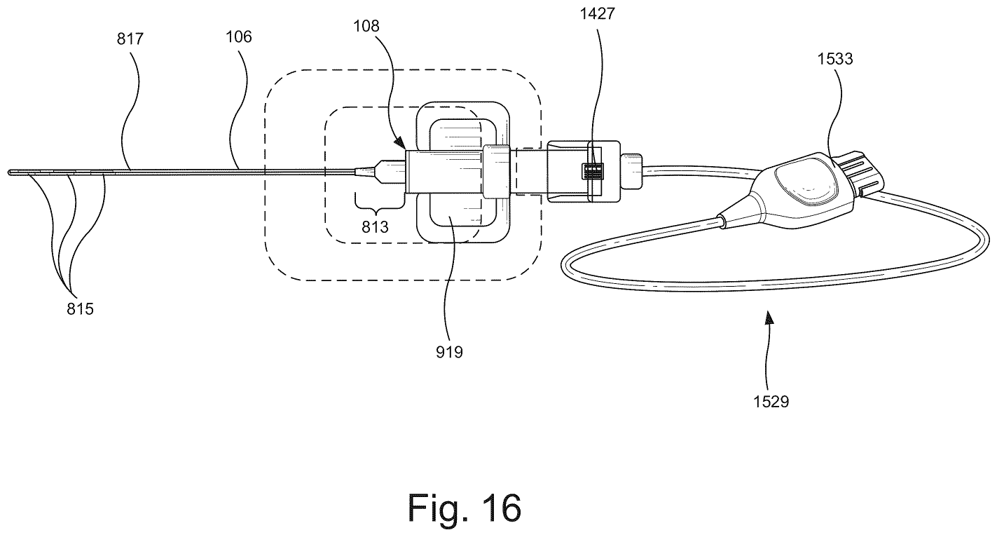

FIG. 16 is a top view of a digital probe 106, data hub 108, and a wired connection 1529 according to some embodiments of the present disclosure. As described in the present disclosure, the vascular probe insertion assembly 100 may include the digital probe 106 operatively coupled to the data hub 108 via a strain relief 813. The digital probe 106 may include one or more sensors 815 operatively coupled to a nitinol core wire 817 to be inserted into a patient's vascular anatomy. The data hub 108 may also include a stabilization platform 919 used to give structural support to the data hub 108 as well as, in an embodiment secure the data hub 108 to the patient's external anatomy via, for example, an adhesive.

As described in the present disclosure, the data hub 108 may include an electrical interface 1427. The electrical interface 1427 may be used by the data hub 108 to couple the data hub 108 to a monitoring system via a wired connection. In some embodiments, the electrical interface 1427 may include one or more contact pins that interface with a corresponding wired connection that operatively couples the data hub 108 to the monitoring system. As described in the present disclosure, the data hub 108 may be a wireless data hub 108 or a wired data hub 108 and the example shown in FIG. 14 is a wired data hub 108 that allows a healthcare professional to connect a wired connection interface to the proximal end of the data hub 108.

FIG. 16 shows the wired connection 1529 described in FIG. 16 being coupled to the electrical interface 1427 and which is used to operatively couple the data hub 108 of the vascular probe insertion assembly 100 to the monitoring system described in the present disclosure. The wired connection 1529 may include a wired connection interface 1531 that is used to interface with the electrical interface 1427 of the data hub 108. Accordingly, the wired connection interface 1531 may include one or more interfacing contact pins that interface with the contact pins of the electrical interface 1427 on the data hub 108 in an embodiment. The wired connection 1529 also may include a plug 1533 used to complete the connection between the data hub 108 and wired connection 1529 to a monitoring system. In some embodiments, the monitoring system may include a bedside monitoring system, a computer or computing device, a data cloud or a combination thereof. In some embodiments, the monitoring system may be used to compute, classify, process, transmit, receive, retrieve, originate, switch, store, display, manifest, detect, record, reproduce, handle, or use any form of information, intelligence, or data for, in an example embodiment, medical diagnosis or other purposes.

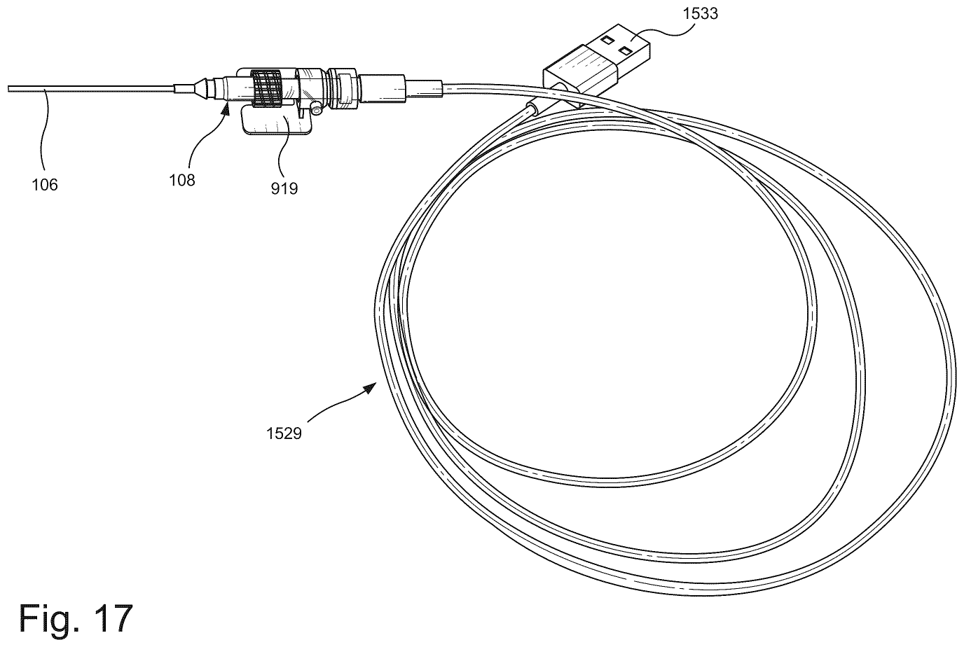

FIG. 17 is a perspective view of a vascular probe insertion assembly 100 with a wired connection 1529 according to some embodiments of the present disclosure. As described in the present disclosure, the data hub 108 may include the digital probe 106 used to place one or more sensors into the vascular anatomy of a patient. The data hub 108 may also include a stabilization platform 919 used to give structural support to the data hub 108 as well as, in an embodiment secure the data hub 108 to the patient's external anatomy via, for example, an adhesive.

In FIG. 17, the data hub 108 is further coupled to a wired connection 1529 used to operatively couple the data hub 108 to a monitoring system via a wired connection. In some embodiments, the wired connection 1529 is permanently coupled to the data hub 108 at a proximal end of the data hub 108. The wired connection 1529 may further include a plug 1533 used to couple the data hub 108 to a monitoring system. In some embodiments, this plug 1533 may be a Universal Serial Bus (USB) type plug that may interface with a USB port at the monitoring system. The length of the wired connection 1529 may vary and may, in one embodiment, be long enough to reach from a patient to the monitoring system.

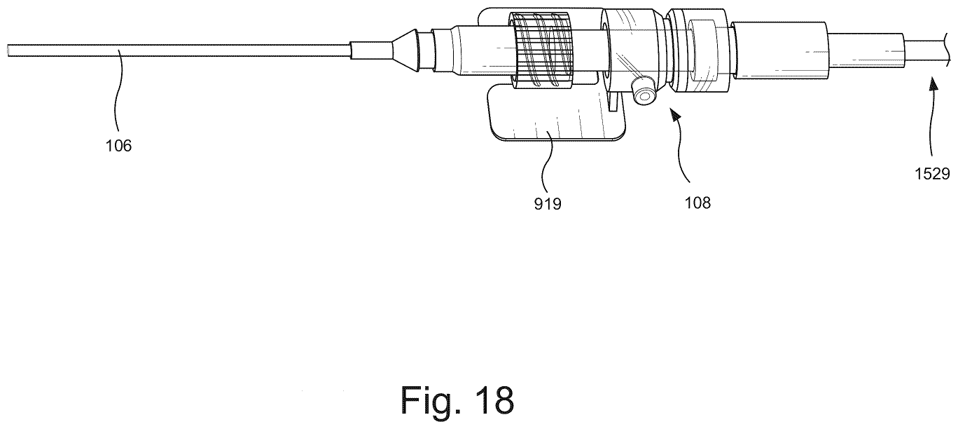

FIG. 18 shows the permanent connection of the wired connection 1529 with the data hub 108. FIG. 18 is a side view of a digital probe 106 with a wired connection 1529 according to some embodiments of the present disclosure. In some embodiments, as the digital probe 106 is placed within the patient's vascular anatomy, the wired connection 1529 allows a healthcare professional may operatively coupled to a plug (not shown) into a monitoring system. In some embodiments, the wired connection 1529 may be disconnected from the digital probe 106 and the stabilization platform 919, if necessary, to allow for better patient mobility. The wired connection 1529 may be subsequently reconnected when those blood based critical patient parameters are to be detected or monitored as described in the present disclosure.

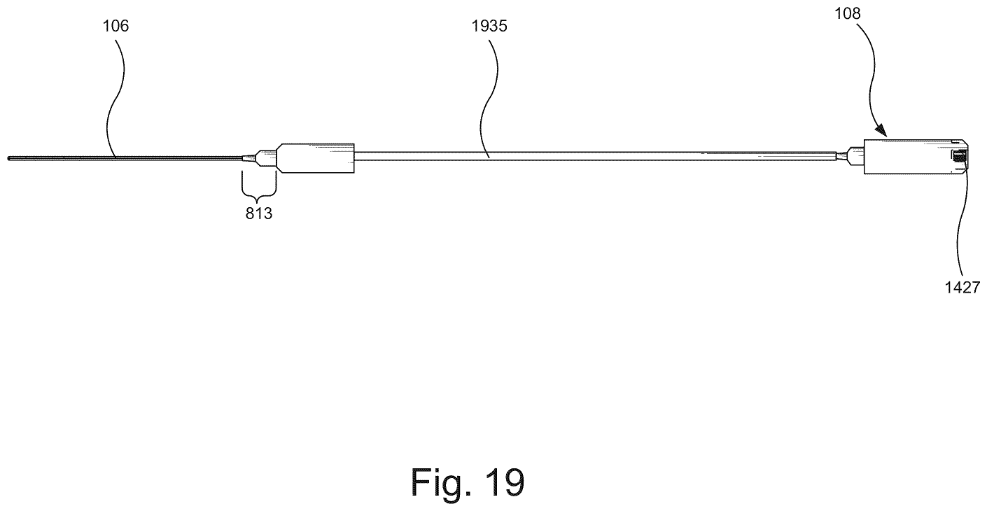

FIG. 19 is a top view of a digital probe 106 and data hub 108 according to some embodiments of the present disclosure. FIG. 19 shows the data hub 108 including a tethered connector 1935 that operatively connects the data hub 108 to the digital probe 106. In some embodiments, the length of the tethered connector 1935 may be varied depending on what level of patient mobility is necessary. In some embodiments, the tethered connector 1935 and data hub 108 may also be secured to the patient's external anatomy to prevent dislodgement of the digital probe 106.

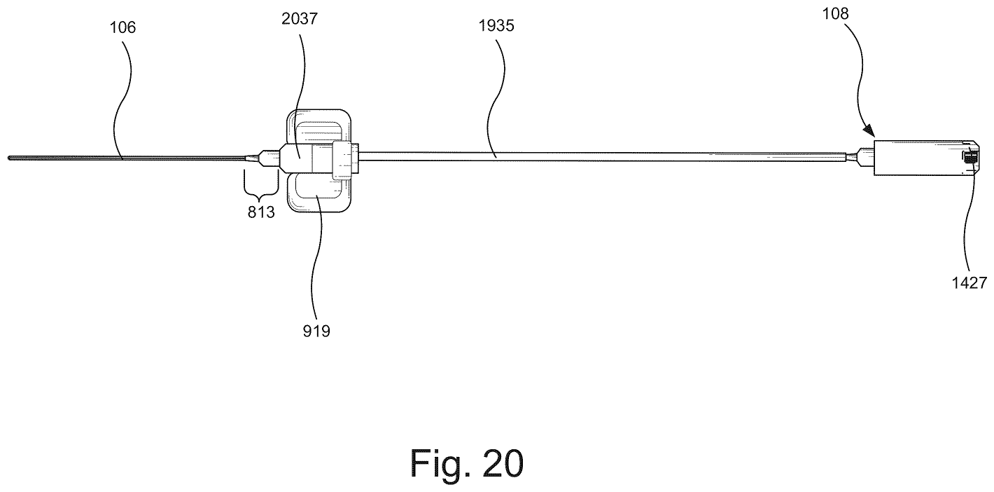

FIG. 20 is a top view of a digital probe 106 and data hub 108 according to some embodiments of the present disclosure. In the embodiment shown in FIG. 20, the data hub 108 may include a tethered connector 1935 that operatively connects the data hub 108 to the digital probe 106. In some embodiments, the length of the tethered connector 1935 may be varied depending on what level of patient mobility is necessary. In some embodiments, the tethered connector 1935 and data hub 108 may also be secured to the patient's external anatomy to prevent dislodgement of the digital probe 106.

In some embodiments, a stabilization platform 919 may be operatively coupled to an intermediary hub 2037 close to the digital probe 106. The stabilization platform 919 may, in some embodiments, be semi rigid that allows the data hub 108 to be placed against the patient's external anatomy so that it does not rotate while the digital probe 106 is placed within the patient's vascular anatomy as described in the present disclosure. In some embodiments, the stabilization platform 919 may include an adhesive formed on a surface that is to abut the patient's external anatomy. In some embodiments, the stabilization platform 919 may include a non-slip surface that creates more friction between the stabilization platform 919 and the patient's external anatomy. In some embodiments, the stabilization platform 919 may reinforce the stability of the intermediary hub 2037 being placed against the patient's external anatomy. In some embodiments, the intermediary hub 2037 may include some of those elements that may be included within the data hub 108. In some embodiments, the intermediary hub 2037 may include one or more of the power source, the PMU, and the microcontroller or other hardware processing device, among other circuitry. Where these devices are included within the intermediary hub 2037, the data hub 108 does not need to include these because the tethered connector 1935 operatively couples the intermediary hub 2037 to the data hub 108.

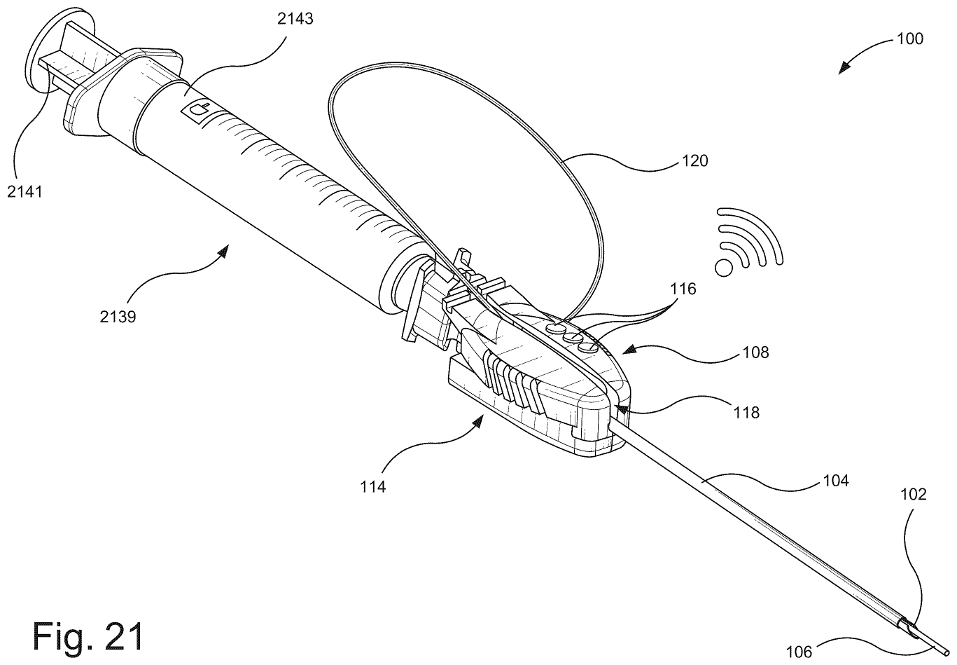

FIG. 21 is a side perspective view of a vascular probe insertion assembly according to some embodiments of the present disclosure. The vascular probe insertion assembly 100 shown in FIG. 21 may be similar to the vascular probe insertion assembly 100 shown in FIG. 1 with a syringe 2139 operatively coupled to the slotted cannula 102.

The vascular probe insertion assembly 100 may include a number of devices used by a healthcare professional to introduce a digital probe 106 into a vein or artery of a patient. The vascular probe insertion assembly 100 may include a slotted cannula 102. The slotted cannula 102 may include slot formed along the length of the slotted cannula 102 into which a digital probe 106 may be placed. The slot formed in the slotted cannula 102 forms sidewalls extending along the length of the slotted cannula 102 with the sidewalls maintaining the digital probe 106 coaxially within except at the slot. The digital probe 106 may, therefore, run coaxially within the slotted cannula 102 until the slotted cannula 102 is removed from the patient's anatomy as described in the present disclosure. In some embodiments, the orientation of the slot formed in the slotted cannula 102 may be aligned with a trench or digital probe channel 118 formed through the data hub 108 so that the digital probe 106 can leave the digital probe channel 118 formed in the data hub 108 when the digital probe 106 is inserted into the patient's vascular anatomy.

The digital probe 106 may include, In some embodiments, a core wire for structure and durability during advancement of the digital probe 106 through the patient's vascular anatomy. The core wire may be a nitinol core wire that runs the length of the digital probe 106 either at the center axis of the digital probe 106 or offset from the center axis. In some embodiments, the digital probe 106 may include optical fibers and/or wires connected to one or more sensors placed along the length of the digital probe 106. In some embodiments, a distal end of the digital probe 106 may include an atraumatic tip that reduces the risk of the digital probe 106 inducing damage or complications in the patient's vein or artery.

In some embodiments, the digital probe 106 may be coated with a variety of coatings used to improve the performance of the digital probe 106 as well as reduce the risk of complications such as thrombus or probe-related blood stream infections (e.g., sepsis). These coatings may include silicon lubes that may or may not include an antimicrobial additive such as alkanes or saturated hydrocarbons (e.g., CH (x)). In some embodiments, the digital probe 106 may be coated with any of anti-thrombogenic or anti-microbial coatings and/or polymer additives.

The sensors present on the digital probe 106 may include any number or type of sensors that detect or measure parameters of a patient's vascular anatomy. These sensors may be placed along the digital probe 106 as individual sensors or bundles of sensors. These sensors may include sensors that incorporate technologies that can detect or measure a patient's blood pressure (venous or arterial), blood gases, blood pH levels, and presence of electrolytes among other types of sensors. In some embodiments, the sensors present on the digital probe 106 may be selected to detect or measure other targeted physiological or procedural parameters.

The vascular probe insertion assembly 100 also may include a splitable sheath 104 formed around the slotted cannula 102. In some embodiments, the splitable sheath 104 may extend the entire length of the slotted cannula 102 except along a beveled portion of the slotted cannula 102. This allows the sharp terminal end of the slotted cannula 102 to penetrate a patient's skin when the healthcare professional accesses the patient's vein or artery. The splitable sheath 104 also prevents the digital probe 106 from exiting the slot formed along the slotted cannula 102 during insertion of the vascular probe insertion assembly 100 into the patient's vascular anatomy.

The vascular probe insertion assembly 100 also may include a needle safety shield 114. The needle safety shield 114 is formed around a portion of the slotted cannula 102 and is allowed to move along the shaft of the slotted cannula 102 during operation. In some embodiments, the needle safety shield 114 has a via formed through it such that the slotted cannula 102 may be passed therethrough. Additionally, the needle safety shield 114, In some embodiments, may include a needle safety shield cutting blade (not shown in FIG. 21) that is used to cut the splitable sheath 104 during extraction of the slotted cannula 102 from the patient's vascular anatomy.

The vascular probe insertion assembly 100 may also include a data hub 108 operatively coupled to the digital probe 106. The data hub 108 may include any type of circuitry used to receive and transmit data detected by the digital probe 106 when the digital probe 106 has been successfully inserted into the patient's vein or artery. In some embodiments, this circuitry may include a wireless radio or other transmitting device that allows for the wireless transmission of the data detected at the digital probe 106 to be transmitted to, for example, a vascular monitoring system that is described in the present disclosure. Additionally, the data hub 108 may include a power source, a power management unit (PMU), and a microcontroller or other hardware processing device, among other circuitry. In some embodiments, the data hub 108 may include one or more visual indicators 116 used to signal to the healthcare professionals that, for example, the data hub 108 is detecting parameters of a patient's vascular anatomy due to the digital probe 106 being properly inserted into the patient's vascular anatomy. These visual indicators 116 may include a variety of differently colored light emitting diodes (LEDs) that may indicate proper used by the microcontroller in the data hub 108 to determine whether the digital probe 106 has been properly, improperly, or sub-optimally inserted into the patient's anatomy.

In the embodiment shown in FIG. 21, the vascular probe insertion assembly 100 also may include a syringe 2139 operatively coupled to the vascular probe insertion assembly 100 and in fluid communication with the slotted cannula 102. Similar to the needle hub described in FIG. 1, the syringe 2139 may be used by the healthcare professional to detect whether the slotted cannula 102 has reached a patient's vein or artery. The healthcare professional may do this by pulling a plunger 2141 out of a barrel 2143 of the syringe 2139. By pulling the plunger 2141 out of the barrel 2143, the healthcare professional may draw an amount of blood in order to determine whether the slotted cannula 102 has reached the patient's vein or artery. Additionally, the syringe 2139 may be used by the healthcare professional to better hold the vascular probe insertion assembly 100 during insertion of the slotted cannula 102. By holding the syringe 2139, the healthcare professional may more easily insert the slotted cannula 102 into the patient's anatomy at those relatively more sensitive or critical insertion sites.

During operation of the vascular probe insertion assembly 100, the healthcare professional may address the patient's anatomy and determine a location where the digital probe 106 may be placed within the patient's vein or artery. Once an appropriate injection site has been determined, the healthcare professional may insert the vascular probe insertion assembly 100 and, specifically, the slotted cannula 102 with its digital probe 106 and the splitable sheath 104 into the patient's anatomy in into a vein or artery. At this point, because the slotted cannula 102 is fluidically coupled with the syringe 2139, the healthcare professional may visually detect that the slotted cannula 102 has reached a vein or artery by gently pulling the plunger 2141 out of the barrel 2143 of the syringe 2139. Because the barrel 2143 is made of a clear plastic, for example, the healthcare professional may visually detect the filling of the barrel 2143.

Once the healthcare professional has detected the presence of blood in the barrel 2143 of the syringe 2139 indicating that the slotted cannula 102 has reached a vein or artery, the healthcare professional may begin to remove the vascular probe insertion assembly 100 from the patient. In some embodiments, the healthcare professional may do this by grasping the syringe 2139 in one hand and the data hub 108/needle safety shield 114 with the other. While holding the data hub 108/needle safety shield 114 in place, the healthcare professional may begin to pull the syringe 2139 away from the patient and the remaining portions of the vascular probe insertion assembly 100. By doing this, the needle safety shield cutting blade (not shown in FIG. 21) of the needle safety shield 114 may begin to cut the splitable sheath 104 as the slotted cannula 102 is pulled through the via formed through the needle safety shield 114. This will continue until the beveled portion of the slotted cannula 102 is safely housed within the via formed through the needle safety shield 114. This process also may include the removal of the data hub 108 from the needle safety shield 114. In some embodiments, the data hub 108 and needle safety shield 114 may be operatively coupled together via a rail system such that they may be slidably removed from each other as the healthcare professional pulls the slotted cannula 102 and splitable sheath 104 through the via formed through the needle safety shield 114.

As the beveled end of the slotted cannula 102 enters the via formed through the needle safety shield 114, the slotted cannula 102 may be prevented from totally being removed from the via in order to prevent the sharp point of the slotted cannula 102 from touching the healthcare professional and potentially harming the healthcare professional. Additionally, the splitable sheath 104, having been cut lengthwise along the length of the slotted cannula 102 may be separated from the slotted cannula 102 and thrown away with the syringe 2139, slotted cannula 102, and needle safety shield 114.

Additionally, as the slotted cannula 102 is pulled out of the patient's vascular anatomy, the digital probe 106 remains. In some embodiments, a supplemental length of digital probe 120 may be advanced further into the patient's vascular anatomy by the healthcare professional as the slotted cannula 102 is inserted into the vascular anatomy. This allows the advancement of the digital probe 106 further into the patient's vascular anatomy prior to the slotted cannula 102 being removed to, for example, cause the digital probe 106 to remain within the vascular anatomy and preventing the slotted cannula 102 from dragging the digital probe 106 back out of the vascular anatomy. Additionally, while the slotted cannula 102 is being pulled out of the patient's vascular anatomy, the digital probe 106/supplemental length of digital probe 120 may exit the slotted cannula 102 due to the splitable sheath 104 being cut and the slot formed in the slotted cannula 102 serving as an exit for the digital probe 106 to be removed from the slotted cannula 102.

When the slotted cannula 102, splitable sheath 104, needle safety shield 114, and syringe 2139 have been removed from the digital probe 106 and data hub 108, the healthcare professional may determine whether the digital probe 106 has been successfully inserted into the patient's vascular anatomy. This may be done by determining visually whether the digital probe 106 appears to be passing into the patient's anatomy and whether the visual indicators 116 on the data hub 108 indicate proper insertion of the digital probe 106. Where the healthcare professional has determined improper insertion of the digital probe 106, the digital probe 106 may be removed by pulling the digital probe 106 from the patient's vascular anatomy and a new insertion site may be determined using a new vascular probe insertion assembly 100. Where the healthcare professional has determined that the digital probe 106 is properly inserted, the healthcare professional may secure the data hub 108 to the exterior portion of the patient's anatomy (e.g., arm) and apply a securement dressing (not shown in FIG. 21) over the data hub 108/supplemental length of digital probe 120 to prevent dislodgement of the digital probe 106. In some embodiments, the healthcare professional may also apply a skin adhesive at the injection site where the slotted cannula 102 entered the patient's body to secure the digital probe 106 to the patient during indwelling of the digital probe 106.

Where the digital probe 106 and data hub 108 are no longer needed by the healthcare professional to detect or measure parameters of a patient's vascular anatomy, the healthcare professional may remove the digital probe 106 and data hub 108 and throw them away. In some embodiments, the healthcare professional may do this by gripping the data hub 108 and pulling on the coupled digital probe 106 away from the patient's body. This allows the digital probe 106 to be pulled out of the vein or artery, through the remaining portions of the patient's anatomy, and out of the hole formed by the slotted cannula 102 during insertion.

The vascular probe insertion assembly 100 described in the present disclosure provides for direct in-vascular measure of hemodynamic and blood based critical patient parameters with a minimum footprint on the patient's body. The vascular probe insertion assembly 100 also allows for continuous or intermittent monitoring without the procedural and workflow difficulties associated with other monitoring systems. Further, the use of the vascular probe insertion assembly 100 described in the present disclosure does not require use of a separate catheter or vascular access device. Still further, risks associated with other systems used to complete the measurements that the vascular probe insertion assembly 100 can accomplish. The vascular probe insertion assembly 100 described in the present disclosure may be used for short term or long-term monitoring of critical vascular-based parameters while the digital probe 106 is in place.

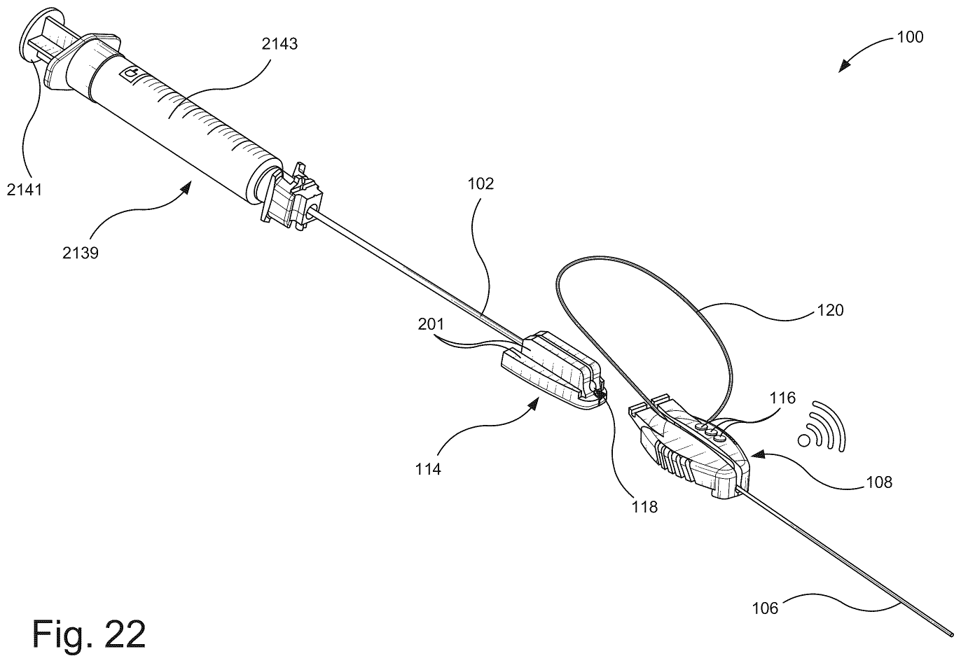

FIG. 22 is a side perspective view of a vascular probe insertion assembly 100 according to some embodiments of the present disclosure. The vascular probe insertion assembly 100 shown in FIG. 22 may be similar to the vascular probe insertion assembly 100 shown in FIG. 21. In the embodiment shown in FIG. 22, the digital probe 106 and data hub 108 have been removed away from the slotted cannula 102, needle safety shield 114 and syringe 2139. The splitable sheath 104 shown in FIG. 21 is not shown in FIG. 22 because, as described in the present disclosure, the movement of the needle safety shield 114 towards a distal end of the slotted cannula 102 where the bevel is formed causes a needle safety shield cutting blade of the needle safety shield 114 to cut the splitable sheath 104 away exposing the slotted cannula 102.

As depicted in FIG. 22, the needle safety shield 114 may include a number of attachment surfaces 201 that interface with complementary attachment surfaces formed on the digital probe 106. The attachment surfaces 201 allow the needle safety shield 114 to be operatively coupled, at least temporarily, to the bottom side of the data hub 108 during insertion of the slotted cannula 102 into the vascular anatomy of the patient. As described in the present disclosure, after the slotted cannula 102 has been inserted into the patient's vein or artery, it may be subsequently removed with the digital probe 106 and data hub 108 remaining in place. In the embodiments in the present disclosure, the removal of the slotted cannula 102 causes the digital probe 106 to remain within the patient's vein or artery while the data hub 108 (operatively coupled to the digital probe 106) remains with the patient. By laterally sliding the needle safety shield 114 away from the data hub 108, the needle safety shield 114, slotted cannula 102, and syringe 2139 are removed away from the data hub 108 thereby separating the needle safety shield 114 from the data hub 108. At this point, the needle safety shield 114, slotted cannula 102, and syringe 2139 may be thrown away or otherwise treated as a biohazard with appropriate disposal steps taken to treat it as such.

The data hub 108 may remain with the patient and later may be allowed to be secured to the outer surface of the patient's anatomy (e.g., arm). As described in the present disclosure, the digital probe 106 may include a supplemental length of digital probe 120 that allows a healthcare professional to advance the digital probe 106 further into the patient's anatomy and into the vein or artery if necessary. This advancement of the digital probe 106 using the supplemental length of digital probe 120 may be accomplished while the slotted cannula 102 is within the vein or artery. Therefore, during removal of the slotted cannula 102, the length of the supplemental length of digital probe 120 may change as the healthcare professional deems is necessary or not to pass the digital probe 106 further into the patient's vein or artery.

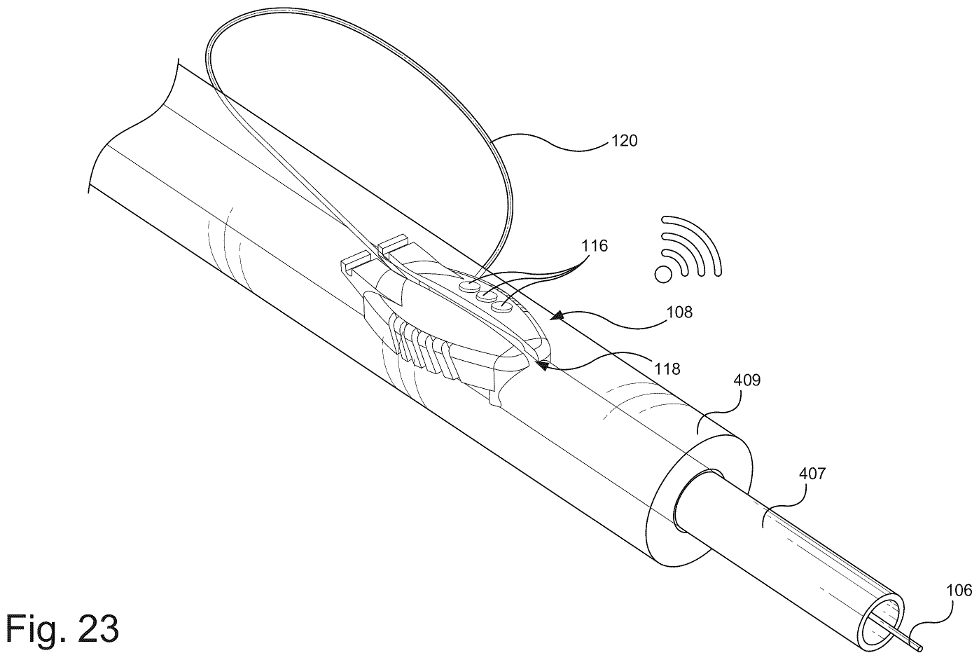

FIG. 23 is a side perspective view of a digital probe 106 inserted into vascular anatomy 407 according to some embodiments of the present disclosure. FIG. 23 shows the digital probe 106 in an installed position after a healthcare professional has removed the slotted cannula, splitable sheath, needle safety shield, and syringe 2139 shown in, for example, FIGS. 21 and 22. As described in the present disclosure, as the slotted cannula is pulled out of the patient's vascular anatomy 407, the digital probe 106 remains. In some embodiments, a supplemental length of digital probe 120 may be advanced further into the patient's vascular anatomy by the healthcare professional after the slotted cannula is inserted into the vascular anatomy 407 but before the slotted cannula is removed from the patient's anatomy. This allows the advancement of the digital probe 106 further into the patient's vascular anatomy 407 prior to the slotted cannula being removed to, for example, cause the digital probe 106 to remain within the vascular anatomy and preventing the slotted cannula from dragging the digital probe 106 back out of the vascular anatomy 407. Additionally, while the slotted cannula is being pulled out of the patient's vascular anatomy 407, the digital probe 106/supplemental length of digital probe 120 may exit the slotted cannula due to the splitable sheath being cut and the slot formed in the slotted cannula serving as an exit for the digital probe 106 to be removed from the slotted cannula.

In some embodiments, the data hub 108 may be secure to the external anatomy 409 of the patient. The external anatomy 409 to which the data hub is secured may vary depending on the vascular anatomy 407 the digital probe 106 is inserted into. For example, where the vascular anatomy 407 is a vein within a patient's arm, the data hub 108 may be secured to the patient's arm close to where the digital probe 106 has passed through the patient's skin.

As described in the present disclosure, the vascular probe insertion assembly 100 may also include a data hub 108 operatively coupled to the digital probe 106. The data hub 108 may include any type of circuitry used to receive and transmit data detected by the digital probe 106 when the digital probe 106 has been successfully inserted into the patient's vein or artery. In some embodiments, this circuitry may include a wireless radio or other transmitting device that allows for the wireless transmission of the data detected at the digital probe 106 to be transmitted to, for example, a vascular monitoring system that is described in the present disclosure. Additionally, the data hub 108 may include a power source, a PMU, and a microcontroller or other hardware processing device, among other circuitry. In some embodiments, the data hub 108 may include one or more visual indicators 116 used to signal to the healthcare professionals that, for example, the data hub 108 is detecting parameters of a patient's vascular anatomy due to the digital probe 106 being properly inserted into the patient's vascular anatomy. These visual indicators 116 may include a variety of differently colored light emitting diodes (LEDs) that may indicate proper used by the microcontroller in the data hub 108 to determine whether the digital probe 106 has been properly, improperly, or sub-optimally inserted into the patient's anatomy. In some embodiments, the data hub 108 may be secured to the external anatomy 409 of the patient using, for example, a transparent film dressing (e.g., 3M® TEGADERM® transparent film dressing) so that the healthcare professional can still view the visual indicators 116 present on the data hub 108. Other securing devices may be used to secure the data hub 108 to the patient's external anatomy 409. Still further, the securing devices may be selectively removable such that a healthcare professional may remove the securing devices either to swap out a new set of securing devices or to eventually remove the digital probe 106 and data hub 108 when monitoring of the patient's vascular anatomy 407 is no longer necessary.

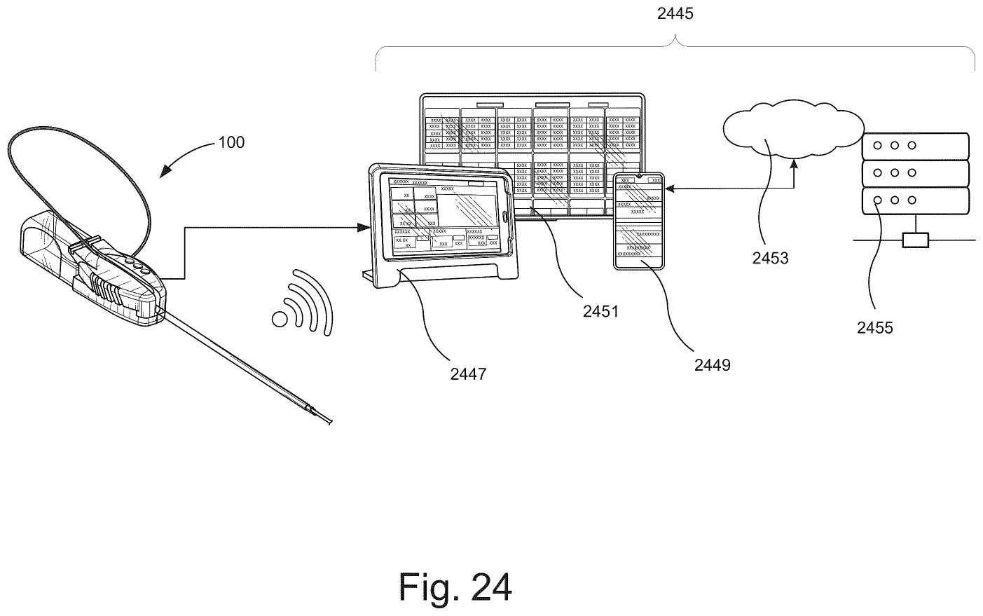

FIG. 24 is a graphic diagram of a vascular probe insertion assembly 100 interfacing with a data processing and cloud-based system 2445 according to some embodiments of the present disclosure. As described in the present disclosure, the vascular probe insertion assembly 100 may be operatively coupled to the data processing and cloud-based system 2445 via a wired or wireless connection. In some embodiments, the vascular probe insertion assembly 100 may be operatively coupled to a patient monitor 2447. In some embodiments, the patient monitor 2447 may be a bedside monitoring system that is present near the patient in a hospital room. This patient monitor 2447 may allow a healthcare professional to see real-time blood based critical patient parameters as detected by the digital probe of the vascular probe insertion assembly 100 when it is installed within the patient's vascular anatomy.

In some embodiments, the vascular probe insertion assembly 100 may additionally or alternatively be operatively coupled to a cell phone 2449 or other hand-held device within the data processing and cloud-based system 2445. Similar to the patient monitor 2447, the cell phone 2449 may provide a healthcare professional with real-time blood based critical patient parameters as detected by the digital probe of the vascular probe insertion assembly 100 when it is installed within the patient's vascular anatomy. In some embodiments, the cell phone 2449 may execute computer readable program code of a monitoring application associated with the vascular probe insertion assembly 100.

In some embodiments, the vascular probe insertion assembly 100 may be operatively coupled to a vital signs monitor 2451 within the data processing and cloud-based system 2445. In some embodiments, the vital signs monitor 2451 may be present at, for example, a nursing station or other remote desktop where a healthcare professional may monitor the blood based critical patient parameters of the patient as detected by the digital probe 106 of the vascular probe insertion assembly 100. It is appreciated that any or all of the patient monitor 2447, cell phone 2449, and/or vital signs monitor 2451 may be used by a healthcare professional to see and monitor the blood based critical patient parameters of the patient as the digital probe is placed within the patient's vascular anatomy. Each of the patient monitor 2447, cell phone 2449, and vital signs monitor 2451 may be coupled to each other for communication between these devices. Still further, each of the patient monitor 2447, cell phone 2449, and vital signs monitor 2451 may be operatively coupled to a cloud network 2453 used by, for example, a hospital to maintain records related to the blood based critical patient parameters of the patient as the digital probe of the vascular probe insertion assembly 100 is placed within the patient's vascular anatomy. In some embodiments, the cloud network 2453 may include a backend server 2455 used to maintain this data.

In some embodiments, the data processing and cloud-based system 2445 may include artificial intelligence (AI) data algorithms and computer readable program code used to detect and predict the onset of complications, therapeutic response, health improvements, and/or changes in disease state of the patient based on the data received at the digital probe of the vascular probe insertion assembly 100. The execution of the computer-readable program code of the AI data algorithms may be completed using a hardware processing device of any of the patient monitor 2447, cell phone 2449, vital signs monitor 2451, and/or backend server 2455 described in the present disclosure. Therefore, any of these devices may be used to detect and predict the onset of complications, therapeutic response, health improvements, and/or changes in disease state of the patient based on the data received at the digital probe of the vascular probe insertion assembly 100. Still further, as any onset of complications, therapeutic response, health improvements, and/or changes in disease state of the patient are detected by execution of the AI data algorithms may elicit a warning (e.g., visible warning, audible warning, etc.) to the healthcare professionals indicating a change in the blood based critical patient parameters as described in the present disclosure.

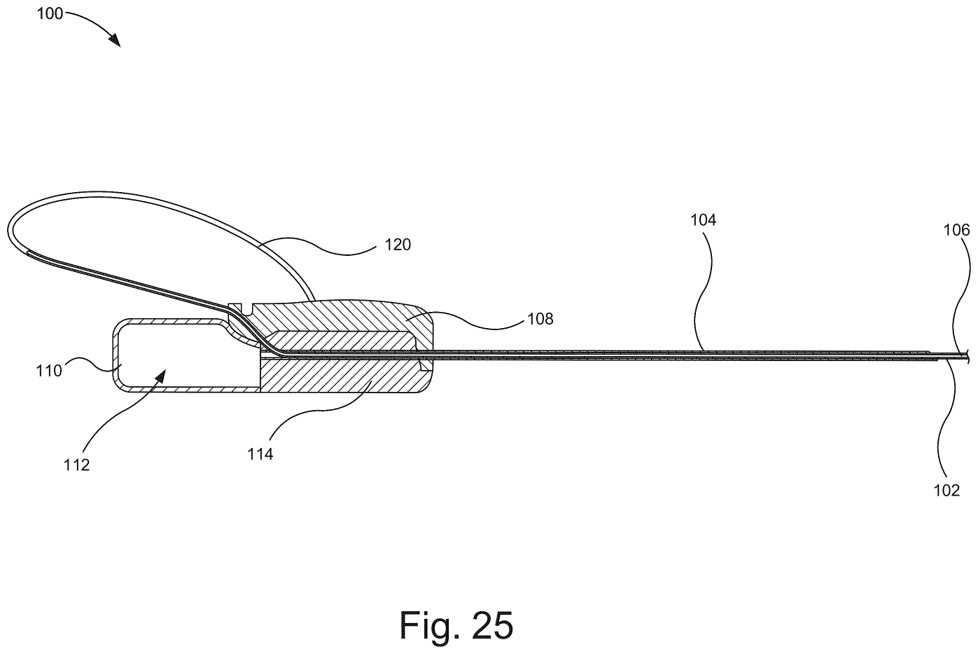

FIG. 25 is a side, cross-sectional, perspective view of a vascular probe insertion assembly 100 according to some embodiments of the present disclosure. FIG. 25 shows the vascular probe insertion assembly 100 cross-sectionally cut along, for example, the digital probe channel (e.g., FIG. 1, 118) showing the digital probe 106 as it passes through the data hub 108 prior to insertion of the slotted cannula 102.

FIG. 25 further shows that the needle hub 110 is in fluid communication with the slotted cannula 102 and the splitable sheath 104. As described in the present disclosure, this allows a healthcare professional to detect when the slotted cannula 102 has been inserted into the patient's vascular anatomy. The healthcare professional detects this by visually monitoring for the presence of blood within the fluid reservoir 112 of the needle hub 110. When blood is detected within the fluid reservoir 112, the healthcare professional may continue by retracting the needle hub 110 away from the data hub 108 and needle safety shield 114 to remove these elements from the vascular anatomy of the patient as well as from the data hub 108 as described in the present disclosure. Additionally, prior to removal of the slotted cannula 102 from the patient's vascular anatomy, the healthcare professional may advance the digital probe 106 further into the patient's vascular anatomy using the supplemental length of digital probe 120. In this embodiment, the healthcare professional may grasp the supplemental length of digital probe 120 and advance it further into the digital probe channel 118 and into the slotted cannula 102/splitable sheath 104 in order to advance the digital probe 106 further into the patient's vascular anatomy. As described in the present disclosure, the healthcare professional may refer to a visual indicator (not shown FIG. 25) on the data hub 108 to determine whether the advancement of the supplemental length of digital probe 120 into the patient's vascular anatomy results in a proper placement of the digital probe 106.

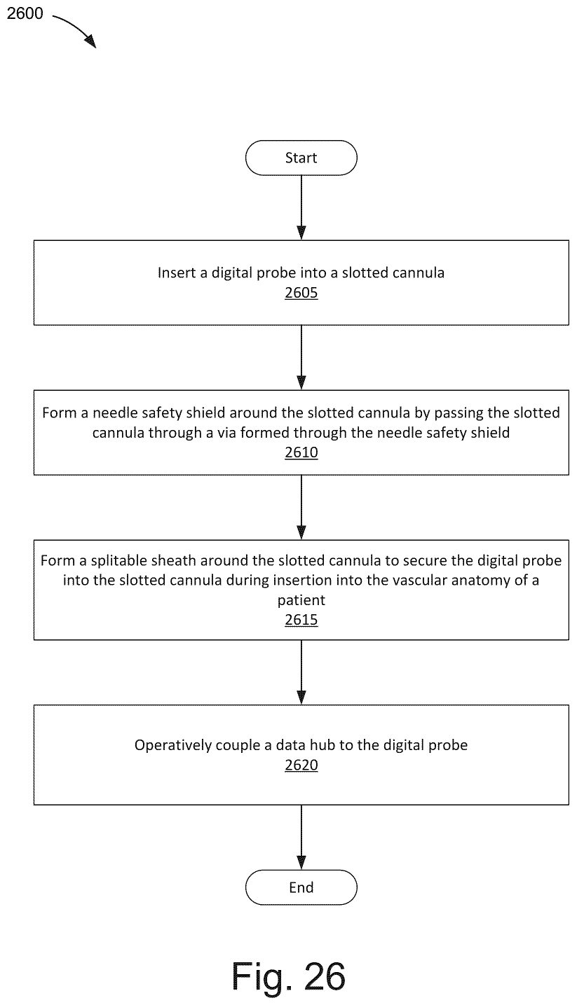

FIG. 26 is a block diagram of a method 2600 of manufacturing a vascular probe insertion assembly according to some embodiments of the present disclosure. The method 2600 may include inserting a digital probe into a slotted cannula at block 2605. As described in the present disclosure, the slot formed in the slotted cannula forms sidewalls extending along the length of the slotted cannula with the sidewalls maintaining the digital probe coaxially within except at the slot. The digital probe may, therefore, run coaxially within the slotted cannula until the slotted cannula is removed from the patient's anatomy as described in the present disclosure.

At block 2610, the method 2600 further may include forming a needle safety shield around the slotted cannula by passing the slotted cannula (and digital probe) through a via formed through the needle safety shield. The needle safety shield is formed around a portion of the slotted cannula and, during operation by a healthcare professional, is allowed to move along the shaft of the slotted cannula. Additionally, the needle safety shield, In some embodiments, may include a needle safety shield cutting blade that is used to cut the splitable sheath during extraction of the slotted cannula from the patient's vascular anatomy.

The method 2600 further may include, at block 2615, forming a splitable sheath around the slotted cannula to secure the digital probe into the slotted cannula during insertion into the vascular anatomy of a patient. As described in the present disclosure, the splitable sheath may be made of a material that can be cut or otherwise split using the needle safety shield cutting blade of the needle safety shield.

The method 2600 further may include operatively coupling a data hub to the digital probe at block 2620. The data hub may include any type of circuitry used to receive and transmit data detected by the digital probe when the digital probe has been successfully inserted into the patient's vein or artery. In some embodiments, this circuitry may include a wireless radio or other transmitting device that allows for the wireless transmission of the data detected at the digital probe to be transmitted to, for example, a vascular monitoring system that is described in the present disclosure. Additionally, the data hub may include a power source, a PMU, and a microcontroller or other hardware processing device, among other circuitry. In some embodiments, the data hub may include one or more visual indicators used to signal to the healthcare professionals that, for example, the data hub is detecting parameters of a patient's vascular anatomy due to the digital probe being properly inserted into the patient's vascular anatomy. These visual indicators may include a variety of differently colored LEDs that may indicate proper placement of the digital probe (e.g., green LED lighted), improper placement of the digital probe (e.g., red LED lighted), or sub-optimal placement of the digital probe (e.g., yellow LED lighted). In some embodiments, the data detected (or not) by the digital probe may be used by the microcontroller in the data hub to determine whether the digital probe has been properly, improperly, or sub-optimally inserted into the patient's anatomy.

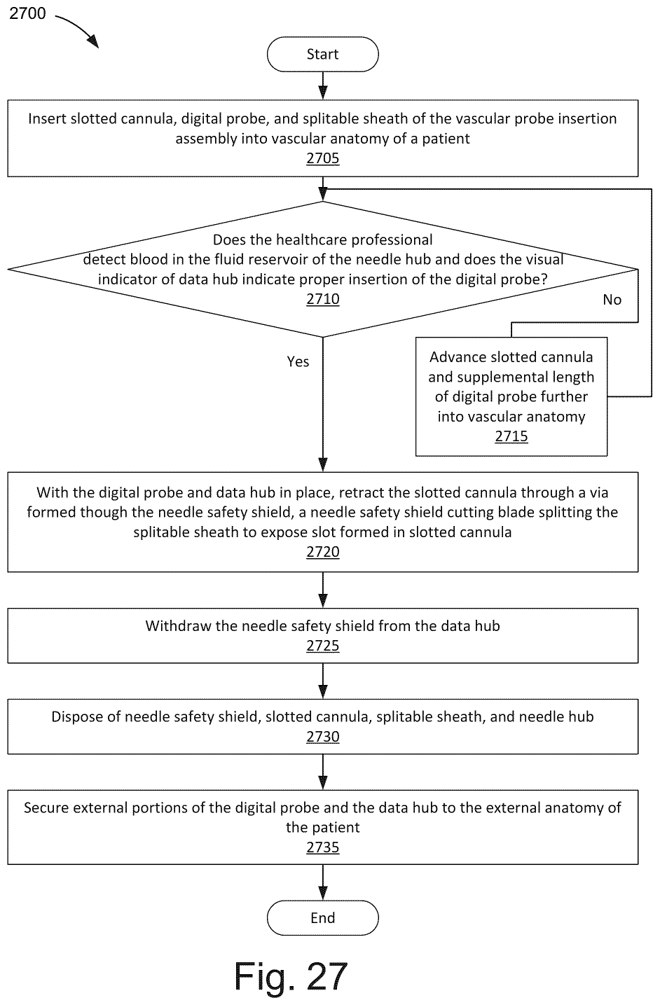

FIG. 27 is a block diagram of a method 2700 of inserting a digital probe of a vascular probe insertion assembly into a patient's anatomy according to some embodiments of the present disclosure. The method 2700 may begin with a healthcare professional inserting the slotted cannula, digital probe, and splitable sheath of the vascular probe insertion assembly into vascular anatomy of a patient at block 2705. This may be done after the healthcare professional has addressed the patient's anatomy in order to determine a location where the digital probe may be placed within the patient's vein or artery. As described in the present disclosure, the vascular probe insertion assembly may include the slotted cannula, digital probe, and splitable sheath that has a certain length. The length of the slotted cannula, digital probe, and splitable sheath inserted into the patient's anatomy and vascular anatomy may depend on the location of insertion on the patient's body.

As described in the present disclosure, the slotted cannula may be fluidically coupled with the fluid reservoir of the needle hub. The method 2700, therefore, may include determining if the healthcare professional detects the presence of blood in a fluid reservoir of a needle hub and whether a visual indicator of the data hub indicates proper insertion at block 2710. Again, the healthcare professional may visually detect that the slotted cannula has reached a vein or artery by the presence of blood within the fluid reservoir. Because the needle hub is made of a clear plastic, for example, the healthcare professional may visually detect the filling of the fluid reservoir. Further, data hub may include one or more visual indicators used to signal to the healthcare professionals that, for example, the data hub is detecting parameters of a patient's vascular anatomy due to the digital probe being properly inserted into the patient's vascular anatomy. These visual indicators may include a variety of differently colored LED) that may indicate proper placement of the digital probe (e.g., green LED lighted), improper placement of the digital probe (e.g., red LED lighted), or sub-optimal placement of the digital probe (e.g., yellow LED lighted). In some embodiments, the data detected (or not) by the digital probe may be used by the microcontroller in the data hub to determine whether the digital probe has been properly, improperly, or sub-optimally inserted into the patient's anatomy.

Where either the healthcare professional has not detected the presence of blood in the fluid reservoir or the visual indicators on the data hub has detected improper or sub-optimal placement of the digital probe (“No” at block 2710), the method 2700 may continue with advancing the slotted cannula (with the digital probe and splitable sheath) and, where available, a supplemental length of digital probe further into the vascular anatomy of the patient at block 2715. The advancement of the cannula into the patient's anatomy to reach the patient's vascular anatomy may be done until the healthcare professional detects blood within the fluid reservoir of the needle hub. It is appreciated, that instead of the needle hub, the vascular probe insertion assembly may include a syringe as described in the present disclosure for the healthcare professional to use to determine whether the slotted cannula has reached the patient's vascular anatomy. Additionally, where the healthcare professional has detected blood within the fluid reservoir (or barrel of the syringe), the healthcare professional may further advance the supplemental length of digital probe into the vascular anatomy of the patient as described in the present disclosure. According to the method 2700, the healthcare professional may continue to monitor for the presence of blood in the needle hub and confirmation of proper digital probe placement via the visual indicators until both are visually detected.

Where the presence of blood is detected and the visual indicators on the data hub indicate proper placement of the digital probe, the method 2700 may include the healthcare professional retracting the slotted cannula through a via formed though the needle safety shield at block 2720. As this occurs as a needle safety shield cutting blade splits the splitable sheath to expose slot formed in slotted cannula as described in the present disclosure. Also, by pulling the splitable sheath and slotted cannula through the via formed in the needle safety shield, a beveled and sharp point of the slotted cannula is retained within the needle safety shield so that this sharp point does not accidentally injure the healthcare professional. Still further, as the healthcare professional pulls the needle safety shield away from the data hub, the needle safety shield is also withdrawn from the data hub at block 2725.

The method 2700 also may include the healthcare professional disposing of the needle safety shield, the slotted cannula, the splitable sheath, and the needle hub at block 2730. Because they have been used, the needle safety shield, slotted cannula, and needle hub may be thrown away or otherwise treated as a biohazard with appropriate disposal steps taken to treat them as such.

The method 2700 further may include securing the external portions of the digital probe and the data hub to the external anatomy of the patient at block 2735. It is appreciated that any securement dressing may be used including those securement dressings that have transparent windows (e.g., 3M® TEGADERM® transparent film dressing) as well as any adhesive stabilization platforms of the data hub.

The vascular probe insertion assembly described in the present disclosure may provide for direct in-vascular measure of hemodynamic and blood based critical patient parameters with a minimum footprint on the patient's body. The vascular probe insertion assembly also allows for continuous or intermittent monitoring without the procedural and workflow difficulties associated with other monitoring systems. Further, the use of the vascular probe insertion assembly described in the present disclosure does not require use of a separate catheter or vascular access device. Still further, risks associated with other systems used to complete the measurements that the vascular probe insertion assembly can accomplish. The vascular probe insertion assembly described in the present disclosure may be used for short term or long-term monitoring of critical vascular-based parameters while the digital probe is in place.

All examples and conditional language recited in the present disclosure are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the disclosed embodiments.

1. A vascular probe insertion assembly, comprising:
a slotted cannula comprising:
a sharpened tip configured to be inserted into a patient's vascular anatomy; and
a sidewall extending proximally from the sharpened tip, the sidewall defining a slot;
a splitable sheath shaped to encircle at least part of the sidewall;
a digital probe comprising:
a sensor tip configured to generate sensor data indicative of operation of the vascular anatomy; and
a shaft extending proximally of the sensor tip; and
a data hub operatively coupled to the digital probe, wherein the data hub is configured to receive the sensor data and convey the sensor data to a monitoring system.

2. The vascular probe insertion assembly of claim 1, further comprising:
the vascular probe insertion assembly further comprising a needle hub operatively coupled to a proximal end of the slotted cannula, the needle hub including a fluid reservoir for a healthcare professional to determine when the slotted cannula has been inserted into the patient's vascular anatomy.

3. The vascular probe insertion assembly of claim 1, further comprising:
a needle safety shield comprising a via through which the slotted cannula may pass, wherein the needle safety shield is operatively coupled with the data hub such that the sharpened tip slides into and is received within the needle safety shield slides when the slotted cannula is removed from the patient's vascular anatomy.

4. The vascular probe insertion assembly of claim 3, further comprising:
a needle safety shield cutting blade formed within the via and positioned to cut the splitable sheath when the vascular probe is retracted from within the patient's vascular anatomy.

5. The vascular probe insertion assembly of claim 1, further comprising:
a wireless transmitter formed within the data hub to wirelessly transmit the sensor data to the monitoring system.

6. The vascular probe insertion assembly of claim 1, further comprising:
an electrical interface configured to receive a wired connection through which the sensor data is transmittable to the monitoring system.

7. The vascular probe insertion assembly of claim 1, further comprising:
a syringe coupling feature configured to secure a proximal end of the slotted cannula to a syringe to facilitate insertion of the slotted cannula into the patient's vascular anatomy.

8. The vascular probe insertion assembly of claim 1, further comprising:
a stabilization platform operatively coupled to the data hub to secure the data hub to an exterior surface of the patient's vascular anatomy as the digital probe is maintained within the patient's vascular anatomy.

9. A method of inserting a digital probe of a vascular probe insertion assembly into a patient's anatomy, comprising:
inserting a slotted cannula into a vascular anatomy of a patient, slotted cannula comprising:
a sharpened tip configured to be inserted into a patient's vascular anatomy;
a sidewall extending proximally from the sharpened tip, the sidewall defining a slot;
a splitable sheath shaped to encircle at least part of the sidewall;
a needle safety shield formed coaxially around the slotted cannula; and
a digital probe operatively coupled to a data hub, the digital probe placed coaxially
within the slotted cannula and splitable sheath;
with the digital probe and data hub in place, retracting the slotted cannula through a via formed though the needle safety shield, a needle safety shield cutting blade splitting the splitable sheath to expose slot formed in slotted cannula; and
withdrawing the needle safety shield from the data hub.

10. The method of claim 9, further comprising:
the vascular probe insertion assembly further comprising a needle hub operatively coupled to a proximal end of the slotted cannula, the needle hub including a fluid reservoir for a healthcare professional to determine when the slotted cannula has been inserted into the patient's vascular anatomy.

11. The method of claim 9, further comprising:
the needle safety shield comprising a needle safety shield cutting blade placed within the via formed through the needle safety shield, the needle safety shield cutting blade to cut the splitable sheath when the slotted cannula is retracted from within the patient's vascular anatomy.

12. The method of claim 9, further comprising:
the data hub comprising a wireless transmitter to wirelessly transmit data received at the digital probe to a vascular monitoring system as the digital probe is maintained within the patient's vascular anatomy.

13. The method of claim 9, further comprising:
the data hub comprising an electrical interface configured to receive a wired connection through which sensor data from the digital probe is transmittable to a monitoring system.

14. The method of claim 9, further comprising:
the vascular probe insertion assembly comprising a syringe coupling feature configured to secure a proximal end of the slotted cannula to a syringe to facilitate insertion of the slotted cannula into the patient's vascular anatomy.

15. The method of claim 9, further comprising:
operatively coupling a stabilization platform to the data hub to secure the data hub to an exterior surface of the patient's anatomy as the digital probe is maintained within the patient's vascular anatomy.

16. An indwelling vascular probe insertion assembly, comprising:
a slotted cannula configured to facilitate insertion of the digital probe into a patient's vascular anatomy, the slotted cannula comprising:
a sharpened tip configured to be inserted into a patient's vascular anatomy; and
a sidewall extending proximally from the sharpened tip, the sidewall defining a slot;
a splitable sheath formed coaxially outside of the slotted cannula;
a digital probe formed coaxially within the slotted cannula, the digital probe comprising:
a sensor tip configured to generate sensor data indicative of operation of the vascular anatomy; and
a shaft extending proximally of the sensor tip;
a data hub operatively coupled to the digital probe; and
a needle hub operative coupled to the slotted cannula, the needle hub including a fluid reservoir to determine when the slotted cannula has been inserted into the patient's vascular anatomy.

17. The indwelling vascular probe insertion assembly of claim 16, further comprising:
a needle safety shield comprising a via through which the slotted cannula may pass, wherein the needle safety shield is operatively coupled with the data hub such that the sharpened tip slides into and is received within the needle safety shield slides when the slotted cannula is removed from the patient's vascular anatomy.

18. The indwelling vascular probe insertion assembly of claim 17, further comprising:
a needle safety shield cutting blade formed within the via formed through the needle safety shield to cut the splitable sheath when the digital probe is retracted from within the patient's vascular anatomy.

19. The indwelling vascular probe insertion assembly of claim 16, further comprising:
a wireless transmitter formed within the data hub to wirelessly transmit the sensor data to a monitoring system.

20. The indwelling vascular probe insertion assembly of claim 16, further comprising:
an electrical interface configured to receive a wired connection through which the sensor data is transmittable to the monitoring system.
